(19) United States
(12) Patent Application Publication
(10) Pub. No.: US 2024/0101640 A1
(43) Pub. Date: Mar. 28, 2024

(54) CD91 POLYPEPTIDE FOR USE IN DETECTING HEMOPEXIN:HEME COMPLEX

(71) Applicant: CSL Behring AG, Bern (CH)

(72) Inventors: Catherine OWCZAREK, Northcote, Victoria (AU); Lisa LINDQVIST, Docklands, Victoria (AU); Kirstee MARTIN, Moonee Ponds, Victoria (AU); Thomas GENTINETTA, Bern (CH)

(21) Appl. No.: 18/042,179

(22) PCT Filed: Aug. 20, 2021
(86) PCT No.: PCT/EP2021/073120

§ 371 Date: Feb. 17, 2023

(30) Foreign Application Priority Data
Aug. 21, 2020 (EP) 20192113.7

(51) Int. Cl. C07K 14/705; G01N 33/68; G01N 33/72

(52) U.S. Cl. CPC C07K 14/70596; G01N 33/6872; G01N 33/721

(57) ABSTRACT

The present invention relates to the use of a CD91 polypeptide for detection and quantification of a hemopexin:heme complex in a biological sample, to the use of a CD91 polypeptide for separation of a hemopexin:heme complex from a biological sample and to the monitoring of a hemolysis treatment using a CD91 polypeptide.

### TECHNICAL FIELD

The present invention relates to the use of a CD91 polypeptide for detection and quantification of a hemopexin:heme complex in a biological sample, to the use of a CD91 polypeptide for separation of a hemopexin:heme complex from a biological sample and to the monitoring of a hemolysis treatment using a CD91 polypeptide.

### BACKGROUND OF THE INVENTION

Hemolysis is characterized by the destruction of red blood cells and is a hall-mark of anemic disorders associated with red blood cell abnormalities, such as enzyme defects, hemoglobinopathies, hereditary spherocytosis, paroxysmal nocturnal hemoglobinuria and spur cell anemia, as well as extrinsic factors such as splenomegaly, autoimmune disorders (e.g., Hemolytic disease of the newborn), genetic disorders (e.g., sickle-cell disease or G6PD deficiency), microangiopathic hemolysis, Gram-positive bacterial infection (e.g., streptococcus, Enterococcus and Staphylococcus), parasite infection (e.g., Plasmodium), toxins and trauma (e.g., burns) (Schaer et. al, Blood, 2013; 121(8):1276-84). Hemolysis is also a common disorder of blood transfusions, particularly massive blood transfusions and in patients using an extracorporeal cardiopulmonary support (Rezogali et. al, J. Cardiothorac. Vasc. Anesth., 2017; 31(2):505-15).

The adverse effects seen in patients with conditions associated with hemolysis are largely attributed to the release of iron and iron-containing compounds, such as hemoglobin (Hb) and heme, from red blood cells (Buehler et. al, Transfusion, 2004; 44(10):1516-30). Under physiological conditions, released hemoglobin is bound by soluble proteins such as haptoglobin and transported to macrophages and hepatocytes (Schaer et. al, Front Physiol., 2014; 5:415 and Alayash et. al, Trends Biotechnol., 2013; 31(1):2-3). However, where the incidence of hemolysis is accelerated and becomes pathological in nature, the buffering capacity of haptoglobin is overwhelmed (Muller-Eberhard et. al, Blood, 1968; 32(5):811-5). As a result, hemoglobin is quickly oxidised to ferri-hemoglobin, which in turn releases free heme (comprising protoporphyrin IX and iron). Whilst heme plays a critical role in several biological processes (e.g., as part of essential proteins such as hemoglobin and myoglobin), free heme is highly toxic. Free heme is a source of redox-active iron, which produces highly toxic reactive oxygen species (ROS) that damages lipid membranes, proteins and nucleic acids (Roumenina et. al, J. Mol. Med., 2016; 22(3):200-13). Heme toxicity is further exacerbated by its ability to intercalate into lipid membranes, where it causes oxidation of membrane components and promotes cell lysis and death. The evolutionary pressure of continuous low-level extracellular Hb/heme exposure has led to compensatory mechanisms that control the adverse effects of free Hb/heme under physiological steady-state conditions and during mild hemolysis. These systems include the release of a group of plasma proteins that bind Hb or heme, including the Hb scavenger haptoglobin (Hp) and the heme scavenger proteins hemopexin (Hpx) and α1-microglobulin.

The predominant heme binding protein in plasma and cerebrospinal fluid is hemopexin. Thereby, hemopexin is the key defense against the deleterious effects of heme on cells. Upon binding of heme by hemopexin in a complex, non-linear but significant relationship, hemopexin:heme complexes are endocytosed by a receptor-mediated mechanism by a variety of cells including hepatocytes, macrophages, and syncytiotrophoblasts, where the complex is degraded in lysosomes. The receptor protein for the hemopexin:heme complex is the CD91 protein (Hvidberg et. al, Blood, 2005; 106(7):2572-9).

Cluster of differentiation 91 (CD91) (Lillis et. al, Physiol Rev., 2008; 88(3); 887-918 and Strickland et. al, Arterioscler. Thromb. Vasc. Biol., 2014; 34(3):487-98), also known inter alia as prolow-density lipoprotein receptor-related protein 1, low density lipoprotein receptor-related protein 1 (short: LRP1), alpha-2-macroglobulin receptor (A2MR) or apolipoprotein E receptor (APOER), was first described by Herz et. al, The EMBO Journal, 1988; 7(13):4119-27). The human preproprotein is a 600 kDa precursor protein which is proteolytically processed by furin to a 515 kDa alpha-chain and a 85 kDa beta-chain (identified by SDS) which are non-covalently associated. The molecular weight of the whole protein based on the primary amino acid sequence is 504,606 kDa. It is expressed on a wide spectrum of cell types including macrophages, hepatocytes, fibroblasts, adipocytes, neurons, vascular smooth muscle cells and syncytiotrophoblasts, most abundantly in vascular smooth muscle cells, hepatocytes and neurons. CD91 orthologs are found in vertebrates, where the nucleotide and amino acid structures have been elucidated for a large number of species.

As a member of the low-density lipoprotein receptor family of proteins (LDLR family), CD91 contains cysteine-rich complement-type ligand binding repeats, epidermal growth factor precursor-type repeats (EGF repeat), β-propeller domains, a transmembrane domain and a cytoplasmic domain. The extracellular domain of CD91 is the alpha-chain, which comprises the 31 cysteine-rich complement-type ligand binding repeats arranged in four “ligand binding domains” with 2, 8, 10, and 11 repeats, respectively (numbered I, II, III and IV). The ligand binding domains bind a variety of ligands including extracellular matrix proteins, growth factors, proteases, protease inhibitor complexes and other proteins involved in lipoprotein metabolism. Of the four domains, domains II and IV bind the majority of the ligands (Kang et. al, Biochem. Cell Biol., 2014; 53:15-23). The cysteines form intramolecular disulfide bonds which are believed to be important for the stability of the ligand-binding domains. The 22 copies of the cysteine-rich EGF repeats flank the ligand-binding domains. The EGF repeats and β-propeller domains serve to release ligands in low pH conditions, such as inside endosomes, with the β-propeller domains postulated to displace the ligand at the ligand binding repeats. The transmembrane domain is the β-chain, which contains a 100 residue cytoplasmic tail. This tail contains two NPxY motifs that are responsible for the function in endocytosis and signal transduction.

CD91 plays a key role in intracellular signaling and endocytosis, which thus implicate it in many cellular and biological processes, including lipid and lipoprotein metabolism, protease degradation, platelet derived growth factor receptor regulation, integrin maturation and recycling, regulation of vascular tone, regulation of blood brain barrier permeability, cell growth, cell migration, inflammation, and apoptosis, as well as diseases such as neurodegenerative diseases, atherosclerosis, and cancer (Lillis et. al, J. Thromb. Haemost., 2005; 3(8):1884-93 and Smith et. al, Exp. Cell Res., 1997; 232:246-54).

The CD91 protein is also involved as a receptor protein in the endocytosis of hemopexin:heme complexes by a receptor-mediated mechanism in a variety of cells including hepatocytes, macrophages and neurons, resulting in cellular heme uptake and lysosomal hemopexin degradation, thus eliminating dangerous heme from the blood. CD91 is known as the receptor for the hemopexin:heme complex with the highest affinity (Hada et. al, Biochim. Biophys. Acta, 2014; 1840(7):2351-60).

Whilst endogenous hemopexin can control the adverse effects of free heme under physiological steady-state conditions, it has little effect in maintaining steady-state heme levels under pathophysicogical conditions, such as those associated with hemolysis, where a high level of heme leads to the depletion of endogenous hemopexin, causing heme-mediated oxidative tissue damage. Studies have shown that hemopexin infusion alleviates heme-induced endothelial activation, inflammation, and oxidative injury in experimental mouse models of hemolytic disorders, such as sickle-cell disease (SCD) and β-thalassemia. Hemopexin administration has also been shown to significantly reduce the level of proinflammatory cytokines and counteract heme-induced vasoconstriction in hemolytic animals (Belcher et. al, Blood, 2014; 123(3):377-90 and Belcher et al, PLoS One, 2018; 13(4):e0196455).

As the complexation of heme released under pathophysicogical conditions may need the additional administration of hemopexin in order to complex heme and compensate its deleterious impact, the detection of hemopexin:heme complex and the determination of the amount thereof in a subject would be of utmost importance.

However, so far no suitable detection agent has been found. While CD91 would be a suitable agent for detecting and quantifying hemopexin:heme complexes in a sample of a subject having a condition associated with hemolysis due to its high affinity for hemopexin:heme complex, the large size of the CD91 protein renders its managing difficult. For example, recombinant production is, in view of the large size of the protein, not possible or only possible under aggravated conditions. On the other hand, mere isolation and purification of the protein do not give enough yield for uncomplicated use as a detection agent. Thus, there exists the need for a detection agent of the hemopexin:heme complex allowing efficient detection of the hemopexin:heme complex and easy, quick and cost-effective production of an efficient detection agent with high yield.

The present invention solves this problem by providing a CD91 polypeptide which binds to the hemopexin:heme complex with high affinity.

### DETAILED DESCRIPTION OF THE INVENTION

In the following, the present invention is described in detail. The features of the present invention are described in individual paragraphs or sections. This, however, does not mean that a feature described in a paragraph or section stands isolated from a feature or features described in other paragraphs or section. Rather, a feature described in a paragraph or section can be combined with a feature or features described in other paragraphs or section. Moreover, the meaning of a feature described in a paragraph or section is also applicable, if the same feature is referred to in another context in another paragraph or section.

The term “comprise/es/ing”, as used herein, is meant to “include or encompass” the disclosed features and further features which are not specifically mentioned. The term “comprise/es/ing” is also meant in the sense of “consist/s/ing of” the indicated features, thus not including further features except the indicated features. Thus, the product of the present invention may be characterized by additional features in addition to the features as indicated.

In a first aspect, the present invention provides the use of a CD91 polypeptide having a length of between 300 and 1000 amino acids and comprising or consisting of ligand binding domain III of CD91 or of a fragment thereof which is capable of binding to a hemopexin:heme complex for the detection of a hemopexin:heme complex in a biological sample.

The invention further provides an in vitro method for detecting a hemopexin:heme complex in a biological sample, comprising incubating the biological sample with a CD91 polypeptide as used in the context of the present invention and detecting the binding between the CD91 polypeptide and the hemopexin:heme complex.

The invention further provides the CD91 polypeptide as used in the context of the present invention for use in a method of treating hemolysis or a condition associated with hemolysis. The invention further provides a kit comprising the CD91 polypeptide as used in the context of the present invention.

The invention further provides a solid support to which the CD91 polypeptide as used in the context of the present invention is linked.

In the following, the invention is discussed.

In the context of the present invention, it has been surprisingly found that the ligand binding domain III of CD91 (LRP1) binds to the hemopexin:heme complex, but not to hemopexin alone (see Example 1). This enables its use for the identification of the hemopexin:heme complex in the blood and other biological samples (see Example 2).

The nucleotide and amino acid sequences of a variety of CD91 proteins of different vertebrates are known in the art. For illustrative purposes only, without being limited thereto, reference is made to the amino acid sequence of human CD91 disclosed herein as SEQ ID NO: 1. The corresponding nucleotide sequence and the amino acid sequence are available from the NCBI (National Centre for Biotechnology Information; National Library of Medicine, Bethesda, MD20894, USA; www.ncbi.nlm.nih.gov). The amino acid sequence for isoform 1 is available under the accession number Q07954.2 (LRP1_HUMAN; prolow-density lipoprotein receptor-related protein 1 or low-density lipoprotein receptor-related protein 1; short=LRP-1). SEQ ID NO:1 is also shown below, wherein the ligand binding domains I, II, III and IV are underlined and appear in the indicated order based on the publication Obermoeller-McCormick et. al, J. Cell Sci., 2001, 114(5):899-908).

| SEQ ID NO: 1 | |
|---|---|
| 1 | mltpplllll pllsalvaaa idapktcspk qfacrdqitc iskgwrcdge rdcpdgsdea |
| 61 | peicpqskaq rcqpnehncl gtelcvpmsr lcngvqdcmd gsdegphcre lqgncsrlgc |
| 121 | qhhcvptldg ptcycnssfq lqadgktckd fdecsvygtc sqlctntdgs ficgcvegyl |
| 181 | lqpdnrscka knepvdrppv lliansqnil atylsgaqvs titptstrqt tamdfsyane |
| 241 | tvcwvhvgds aaqtqlkcar mpglkgfvde htinislslh hveqmaidwl tgnfyfvddi |
| 301 | ddrifvcnrn gdtcvtlldl elynpkgial dpamgkvfft dygqipkver cdmdgqnrtk |
| 361 | lvdskivfph gitldlvsrl vywadayldy ievvdyegkg rqtiiqgili ehlygltvfe |
| 421 | nylyatnsdn anaqqktsvi rvnrfnstey qvvtrvdkgg alhiyhqrrq prvrshacen |
| 481 | dqygkpggcs dicllanshk artcrersgf slgsdgksck kpehelflvy gkgrpgiirg |
| 541 | mdmgakvpde hmipienlmn praldfhaet gfiyfadtts yligrqkidg teretilkdg |
| 601 | ihnvegvavd wmgdnlywtd dgpkktisva rlekaaqtrk tliegkmthp raivvdplng |
| 661 | wmywtdweed pkdsrrgrle rawmdgshrd ifvtsktvlw pnglsldipa grlywvdafy |
| 721 | drietillng tdrkivyegp elnhafglch hgnylfwtey rsgsvyrler gvggapptvt |
| 781 | llrserppif eirmydaqqq qvgtnkcrvn nggcsslcla tpgsrqcaca edqvldadgv |
| 841 | tclanpsyvp ppqcqpgefa cansrciger wkcdgdndcl dnsdeapalc hqhtcpsdrf |
| 901 | kcennrcipn rwlcdgdndc gnsedesnat csartcppnq fscasgrcip iswtcdlddd |
| 961 | cgdrsdesas cayptcfplt qftcnngrci ninwrcdndn dcgdnsdeag cshscsstqf |
| 1021 | kcnsgrcipe hwtcdgdndc gdysdethan ctnqatrppg gchtdefqcr ldglciplrw |
| 1081 | rcdgdtdcmd ssdekscegv thvcdpsvkf gckdsarcis kawvcdgdnd cednsdeenc |
| 1141 | eslacrppsh pcanntsvcl ppdklcdgnd dcgdgsdege lcdqcslnng gcshncsvap |
| 1201 | gegivcscpl gmelgpdnht cqiqsycakh lkcsqkcdqn kfsvkcscye gwvlepdges |
| 1261 | crsldpfkpf iifsnrheir ridlhkgdys vlvpglrnti aldfhlsqsa lywtdvvedk |
| 1321 | iyrgklldng altsfevviq yglatpegla vdwiagniyw vesnldqiev akldgtlrtt |
| 1381 | llagdiehpr aialdprdgi lfwtdwdasl prieaasmsg agrrtvhret gsggwpnglt |
| 1441 | vdylekrilw idarsdaiys arydgsghme vlrgheflsh pfavtlygge vywtdwrtnt |
| 1501 | lakankwtgh nvtvvqrtnt qpfdlqvyhp srqpmapnpc eanggqgpcs hlclinynrt |
| 1561 | vscacphlmk lhkdnttcye fkkfllyarq meirgvdlda pyynyiisft vpdidnvtvl |
| 1621 | dydareqrvy wsdvrtqaik rafingtgve tvvsadlpna hglavdwvsr nlfwtsydtn |
| 1681 | kkqinvarld gsfknavvqg leqphglvvh plrgklywtd gdnismanmd gsnrtllfsg |
| 1741 | qkgpvglaid fpesklywis sgnhtinrcn ldgsglevid amrsqlgkat alaimgdklw |
| 1801 | wadqvsekmg tcskadgsgs vvlrnsttlv mhmkvydesi qldhkgtnpc svnngdcsql |
| 1861 | clptsettrs cmctagyslr sgqqacegvg sfllysvheg irgipldpnd ksdalvpvsg |
| 1921 | tslavgidfh aendtiywvd mglstisrak rdqtwredvv tngigrvegi avdwiagniy |
| 1981 | wtdqgfdvie varlngsfry vvisqgldkp raitvhpekg ylfwtewgqy priersrldg |
| 2041 | tervvlvnvs iswpngisvd yqdgklywcd artdkierid letgenrevv lssnnmdmfs |
| 2101 | vsvfedfiyw sdrthangsi krgskdnatd svplrtgigv qlkdikvfnr drqkgtnvca |
| 2161 | vanggcqqlc lyrgrgqrac acahgmlaed gascreyagy llysertilk sihlsdernl |
| 2221 | napvqpfedp ehmknviala fdyragtspg tpnriffsdi hfgniqqind dgsrritive |
| 2281 | nvgsveglay hrgwdtlywt syttstitrh tvdqtrpgaf eretvitmsg ddhprafvld |
| 2341 | ecqnlmfwtn wneqhpsimr aalsganvlt liekdirtpn glaidhraek lyfsdatldk |
| 2401 | ierceydgsh ryvilksepv hpfglavyge hifwtdwvrr avqrankhvg snmkllrvdi |
| 2461 | pqqpmgiiav andtnscels pcrinnggcq dlcllthqgh vncscrggri lqddltcrav |
| 2521 | nsscraqdef ecangecinf sltcdgvphc kdksdekpsy cnsrrckktf rqcsngrcvs |
| 2581 | nmlwcngadd cgdgsdeipc nktacgvgef rcrdgtcign ssrcnqfvdc edasdemncs |
| 2641 | atdcssyfrl gvkgvlfqpc ertslcyaps wvcdgandcg dysderdcpg vkrprcplny |
| 2701 | facpsgrcip mswtcdkedd cehgedethc nkfcseaqfe cqnhrciskq wlcdgsddcg |
| 2761 | dgsdeaahce gktcgpssfs cpgthvcvpe rwlcdgdkdc adgadesiaa gclynstcdd |
| 2821 | refmcqnrqc ipkhfvcdhd rdcadgsdes peceyptcgp sefrcangrc lssrqwecdg |
| 2881 | endchdqsde apknphctsq ehkcnassqf lcssgrcvae allcngqddc gdssdergch |
| 2941 | ineclsrkls gcsqdcedlk igfkcrcrpg frlkddgrtc advdecsttf pcsqrcinth |
| 3001 | gsykclcveg yaprggdphs ckavtdeepf lifanryylr klnldgsnyt llkqglnnav |
| 3061 | aldfdyreqm iywtdvttqg smirrmhlng snvqvlhrtg lsnpdglavd wvggnlywcd |
| 3121 | kgrdtievsk lngayrtvlv ssglrepral vvdvqngyly wtdwgdhsli grigmdgssr |
| 3181 | svivdtkitw pngltldyvt eriywadare dyiefasldg snrhvvlsqd iphifaltlf |
| 3241 | edyvywtdwe tksinrahkt tgtnktllis tlhrpmdlhv fhalrqpdvp nhpckvnngg |
| 3301 | csnlcllspg gghkcacptn fylgsdgrtc vsnctasqfv ckndkcipfw wkcdteddcg |
| 3361 | dhsdeppdcp efkcrpgqfq cstgictnpa ficdgdndcq dnsdeancdi hvclpsqfkc |
| 3421 | tntnrcipgi frcngqdncg dgederdcpe vtcapnqfqc sitkrciprv wvcdrdndcv |
| 3481 | dgsdepanct qmtcgvdefr ckdsgrcipa rwkcdgeddc gdgsdepkee cdertcepyq |
| 3541 | frcknnrcvp grwqcdydnd cgdnsdeesc tprpcsesef scangrciag rwkcdgdhdc |
| 3601 | adgsdekdct prcdmdqfqc ksghciplrw rcdadadcmd gsdeeacgtg vrtcpldefq |
| 3661 | cnntlckpla wkcdgeddcg dnsdenpeec arfvcppnrp frckndrvcl wigrqcdgtd |
| 3721 | ncgdgtdeed cepptahtth ckdkkeflcr ngrclssslr cnmfddcgdg sdeedcsidp |
| 3781 | kltscatnas icgdearcvr tekaaycacr sgfhtvpgqp gcqdineclr fgtcsqlcnn |
| 3841 | tkgghlcsca rnfmkthntc kaegseyqvl yiaddneirs lfpghphsay eqafqgdesv |
| 3901 | ridamdvhvk agrvywtnwh tgtisyrslp paappttsnr hrrqidrgvt hlnisglkmp |
| 3961 | rgiaidwvag nvywtdsgrd vievaqmkge nrktlisgmi dephaivvdp lrgtmywsdw |
| 4021 | gnhpkietaa mdgtlretlv qdniqwptgl avdyhnerly wadaklsvig sirlngtdpi |
| 4081 | vaadskrgls hpfsidvfed yiygvtyinn rvfkihkfgh splvnltggl shasdvvlyh |
| 4141 | qhkqpevtnp cdrkkcewlc llspsgpvct cpngkrldng tcvpvpsptp ppdaprpgtc |
| 4201 | nlqcfnggsc flnarrqpkc rcqprytgdk celdqcwehc rnggtcaasp sgmptcrcpt |
| 4261 | gftgpkctqq vcagycanns tctvnqgnqp qcrclpgflg drcqyrqcsg ycenfgtcqm |
| 4321 | aadgsrqcrc tayfegsrce vnkcsrcleg acvvnkqsgd vtenctdgrv apscltcvgh |
| 4381 | csnggsctmn skmmpecqcp phmtgprcee hvfsqqqpgh iasiliplll llllvlvagy |
| 4441 | vfwykrrvqg akgfqhqrmt ngamnveign ptykmyegge pddvggllda dfaldpdkpt |
| 4501 | nftnpvyatl ymgghgsrhs lastdekrel lgrgpedeig dpla |

As used herein, the term “CD91” or “CD91 protein” refers to the full-length protein, either in the precursor form or in the mature form. It consists of 4,544 amino acids in the precursor form and of 4,525 amino acids in the mature form. Amino acids 1 to 19 are the signal sequence, amino acids 20 to 4419 are the extracellular sequence, amino acids 4420 to 4444 are the transmembrane region and amino acids 4445 to 4544 are the cytoplasmic tail.

The term “CD91 polypeptide”, as referred to herein, refers to any polypeptide portion of precursor or mature CD91, but not to the full-length precursor or mature CD91, which binds to a hemopexin-heme complex, but does not bind to hemopexin to which heme is not bound.

According to the claims, the CD91 polypeptide has a length of between 300 and 1000 amino acids.

The CD91 polypeptide used in the context of the present invention may in particular have a length of 400, 500, 600, 700, 800 or 900 amino acids. It is also envisaged that it may have any length between 400 and 900 amino acids, between 500 and 900 amino acids, between 500 and 800 amino acids, between 600 and 900 amino acids or between 600 and 800 amino acids.

The CD91 polypeptide used in the context of the present invention may comprise sequences derived from CD91 and further peptide sequences.

Preferably, the CD91 polypeptide comprises or consists of a fragment within amino acids 2481 to 2942 of SEQ ID NO: 1, whereby the start of this fragment is at the most 100, 70, 50, 30, 20, 10 or 5 amino acids away from amino acid 2481 and/or amino acid 2942.

The CD91 polypeptide used in the context of the present invention may comprise various parts of CD91. It may, for example, contain an amino acid sequence which is located at the N terminus of CD91 and ligand binding domain III, i.e. amino acids 2481 to 2942.

Unless indicated otherwise, the amino acid numbering of CD91 polypeptide residues in this application refers to SEQ ID NO:1, even if the CD91 molecule does not need to comprise all residues of SEQ ID NO:1.

More preferably, the CD91 polypeptide comprises or consists of amino acids 2481 to 2942 of SEQ ID NO: 1, shown in SEQ ID NO: 2 below or a corresponding amino acid sequence of a homologous CD91. SEQ ID NO:2 denotes ligand binding domain III of CD91. SEQ ID NO:2 has a length of 462 amino acids.

SEQ ID NO: 2:
PCRINNGGCQDLCLLTHQGHVNCSCRGGRILQDDLTCRAVNSSCRAQDE
FECANGECINFSLTCDGVPHCKDKSDEKPSYCNSRRCKKTFRQCSNGRC
VSNMLWCNGADDCGDGSDEIPCNKTACGVGEFRCRDGTCIGNSSRCNQF
VDCEDASDEMNCSATDCSSYFRLGVKGVLFQPCERTSLCYAPSWVCDGA
NDCGDYSDERDCPGVKRPRCPLNYFACPSGRCIPMSWTCDKEDDCEHGE
DETHCNKFCSEAQFECQNHRCISKQWLCDGSDDCGDGSDEAAHCEGKTC
GPSSFSCPGTHVCVPERWLCDGDKDCADGADESIAAGCLYNSTCDDREF
MCQNRQCIPKHFVCDHDRDCADGSDESPECEYPTCGPSEFRCANGRCLS
SRQWECDGENDCHDQSDEAPKNPHCTSQEHKCNASSQFLCSSGRCVAEA
LLCNGQDDCGDSSDERGCHIN

In an even more preferred embodiment, the CD91 polypeptide used according to the present invention comprises the amino acids of SEQ ID NO:2 and further comprises at the N-terminus a signal sequence for secretion of the CD91 polypeptide into the culture medium, preferably the signal sequence of SEQ ID NO: 1, a linker, preferably AIDAP (SEQ ID NO: 5), a tag for CD91 polypeptide purification, preferably the Flag-tag, as known in the art (DYKDDDDKK; SEQ ID NO: 6), and at the C-terminus a tag for CD91 polypeptide purification, preferably a His-tag. A preferred CD91 polypeptide is shown below in SEQ ID NO:3. This construct has been used in example 2 below.

SEQ ID NO: 3:
MLTPPLLLLLPLLSALVAAAIDAPDYKDDDDKKPCRINNGGCQDLCLLT
HQGHVNCSCRGGRILQDDLTCRAVNSSCRAQDEFECANGECINFSLTCD
GVPHCKDKSDEKPSYCNSRRCKKTFRQCSNGRCVSNMLWCNGADDCGDG
SDEIPCNKTACGVGEFRCRDGTCIGNSSRCNQFVDCEDASDEMNCSATD
CSSYFRLGVKGVLFQPCERTSLCYAPSWVCDGANDCGDYSDERDCPGVK
RPRCPLNYFACPSGRCIPMSWTCDKEDDCEHGEDETHCNKFCSEAQFEC
QNHRCISKQWLCDGSDDCGDGSDEAAHCEGKTCGPSSFSCPGTHVCVPE
RWLCDGDKDCADGADESIAAGCLYNSTCDDREFMCQNRQCIPKHFVCDH
DRDCADGSDESPECEYPTCGPSEFRCANGRCLSSRQWECDGENDCHDQS
DEAPKNPHCTSQEHKCNASSQFLCSSGRCVAEALLCNGQDDCGDSSDER
GCHINHHHHHHHH

The skilled person will also appreciate the CD91 polypeptide used in the context of the present invention could even be longer. For example, it could also have a length of 1100, 1200, 1300, 1400, 1600 or even 2000 or 2500 amino acids.

The polypeptide of the invention may further comprise parts of the CD91 sequence which are adjacent to amino acids 2481 to 2942, i.e. adjacent to ligand binding domain III. It may even encompass parts of ligand binding domains II and IV. In this context, adjacent means that the corresponding amino acid sequence ends at position 2480 or starts at position 2943.

The CD91 polypeptide used in the context of the present invention may further comprise ligand binding domain III, i.e. amino acids 2481 to 2942 and additionally peptide stretches which are located between ligand binding domain III and II and/or between ligand binding domain III and IV. Preferably, the CD91 polypeptide may comprise a continuous stretch of amino acids from CD91 which starts after the end of ligand binding domain II (i.e. amino acid 1183) and ends before the beginning of ligand binding domain IV (i.e. amino acid 3293). Possibly, the CD91 polypeptide comprises a continuous stretch of amino acids from CD91 which starts at position 1184 and/or ends at position 3292 and comprises amino acids 2481 to 2942.

The polypeptide of the invention may preferably be glycosylated.

The term “ligand binding domain” refers to four defined regions within the CD91 protein, called ligand binding domains I, II, III and IV. The ligand binding domains are composed of cysteine-rich complement-type ligand binding repeats, arranged in the four ligand binding domains with 2 repeats within ligand binding domain I, 8 repeats within ligand binding domain II, 10 repeats within ligand binding domain III and 11 repeats within ligand binding domain IV, which bind a series of different ligands including extracellular matrix proteins, growth factors, proteases, protease inhibitor complexes and other proteins involved in lipoprotein metabolism. Of the four domains, domains II and IV bind the majority of the ligands, whereby only very few ligands have been detected so far for ligand binding domain III (eg. FVIIIa). Other ligands include apoE-enriched β-VLDL and Lactoferrin (Croy et. al, Biochemistry, 2004; 43(23):7328-35.

CD91 homologs are found in vertebrates. Therefore, the term “CD91” or “CD91 protein”, as referred to herein, refers to any CD91 ortholog or homolog found in vertebrates. Alternatively, CD91, as referred to herein, refers to any CD91 which has an amino acid identity to the sequence of SEQ ID NO: 1 of at least 60%, 70%, 80%, 85%, 90%, 95%, 96%, 97%, 98%, 99% or 100%. Alternatively, the CD91, as referred to herein, refers to any CD91 which has an amino acid homology to SEQ ID NO: 1 of at least 50%, 60%, 70%, 80%, 85%, 90%, 95%, or 100%. Preferably, CD91, as referred to herein, including any CD91 found in vertebrates and any CD91 having an amino acid identity or homology to the sequence of SEQ ID NO: 1, as defined above, has the same activity of the CD91 according to SEQ ID NO: 1. More preferably, the CD91, as referred to herein, binds a hemopexin:heme complex, preferably a human hemopexin-human heme complex, with the same affinity as the CD91 of SEQ ID NO: 1 or with an affinity that is at least 70%, 80%, 85%, 90%, 95%, 96%, 97%, 98%, 99% or 100% of the affinity of CD91 of SEQ ID NO: 1 to a human hemopexin-human heme complex. CD91 with the above definitions of relationship, homology, identity and function is referred to herein also as “CD91 homolog”.

A CD91 polypeptide is a portion of CD91. The term “CD91 polypeptide” is defined above. Thereby, the term “CD91 polypeptide”, as referred to herein, refers to a CD91 polypeptide being a portion of any CD91, as defined above, including CD91 orthologs or homologs found in vertebrates and CD91 defined by its amino acid identity or homology to SEQ ID NO: 1, as indicated above. Alternatively, a CD91 polypeptide has an amino acid identity to the sequence of SEQ ID NO: 2 or comprises an amino acid sequence with an amino acid identity to the sequence of SEQ ID NO:2 of at least 60%, 70%, 80%, 85%, 90%, 95%, 96%, 97%, 98%, 99% or 100 or an amino acid homology to SEQ ID NO: 2 of at least 50%, 60%, 70%, 80%, 85%, 90%, 95%, or 100%. Alternatively, a CD91 polypeptide has an amino acid identity to the sequence of SEQ ID NO: 3 or comprises an amino acid sequence with an amino acid identity to the sequence of SEQ ID NO:3 of at least 60%, 70%, 80%, 85%, 90%, 95%, 96%, 97%, 98%, 99% or 100 or an amino acid homology to SEQ ID NO: 3 of at least 50%, 60%, 70%, 80%, 85%, 90%, 95%, or 100%. Preferably, a CD91 polypeptide, as referred to herein, has the same activity as a CD91 polypeptide derived from SEQ ID NO: 1 or as the CD91 polypeptide according to SEQ ID NO: 2, or 3, respectively. More preferably, a CD91 polypeptide binds a hemopexin:heme complex, preferably a human hemopexin-human heme complex. Binding may be with the same affinity as a CD91 polypeptide derived from SEQ ID NO: 1 or as the CD91 polypeptide of SEQ ID NO: 2, or 3 or with an affinity that is at least 70%, 80%, 85%, 90%, 95%, 96%, 97%, 98%, 99% or 100% of the affinity, with which the CD91 polypeptide derived from SEQ ID NO: 1 or the CD91 polypeptide of SEQ ID NO: 2, or 3 binds to a hemopexin:heme complex, preferably a human hemopexin-human heme complex.

The CD91 polypeptide of the invention binds preferably hemopexin:heme with a Kd of lower than 100 μM, lower than 50 μM, lower than 10 μM, or lower than 5 μM. Preferably, the Kd is lower than 4 μM or lower than 2 μM. The Kd may be higher than 0.1 μM or may be higher than 0.01 μM.

As shown in the examples, the affinity of a ligand binding domain III to the hemopexin:heme complex may be about 1.5 μM or about 2 μM.

The percentage of sequence identity refers to the percentage of amino acid residues which are identical in corresponding positions in two optimally aligned sequences. It is determined by comparing two optimally aligned sequences over a comparison window, where the part of the amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (Add. APL Math, 1981; 2:482-9), by the homology alignment algorithm of Needleman and Wunsch (J. Mol. Biol, 1970; 48:443-53), by the search for similarity method of Pearson and Lipman (PNAS, 1988; 85:2444-8), by the algorithm of Karlin and Altschul (PNAS, 1990; 87:2264-8) modified by Karlin and Altschul (PNAS, 1993; 90:5873-7), or by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by inspection. GAP and BESTFIT are preferably employed to determine the optimal alignment. Typically, the default values of 5.00 for gap weight and 0.30 for gap weight length are used.

The percentage of sequence homology refers to the percentage of amino acid residues which are homologous in corresponding positions in two optimally aligned sequences. The “percentage of homology” between two sequences is established in a manner substantially identical to what has been described above with reference to the determination of the “percentage of identity” except for the fact that in the calculation also homologous positions and not only identical positions are considered. Two homologous amino acids have two identical or homologous amino acids. Homologous amino acid residues have similar chemical-physical properties, for example, amino acids belonging to a same group: aromatic (Phe, Trp, Tyr), acid (Glu, Asp), polar (Gln, Asn), basic (Lys, Arg, His), aliphatic (Ala, Leu, lie, Val), with a hydroxyl group (Ser, Thr), or with a short lateral chain (Gly, Ala, Ser, Thr, Met). It is expected that substitutions between such homologous amino acids do not change a protein phenotype (conservative substitutions).

Moreover, CD91 polypeptide, as referred to herein, may refer to one homolog of a CD91 polypeptide or to a mixture of CD91 polypeptide homologs derived from more than one individual of same species and/or derived from more than one individual of different species, as long as the CD91 polypeptide binds to a hemopexin-heme complex.

Where a hemopexin-heme complex is to be detected in a human, it is generally preferable that the CD91 polypeptide is derived from a human CD91. However, it is to be understood that a CD91 polypeptide derived from a non-human homolog of CD91 may be used for detection of a hemopexin-heme complex in human, as long as the non-human homolog of the CD91 polypeptide has the ability to bind to the human hemopexin-heme complex. Otherwise, a CD91 polypeptide derived from a human CD91 or non-human homolog of CD91 may be used for detection of a hemopexin-heme complex in a non-human being, whereby the non-human homolog of the CD91 polypeptide may be of same or other species, as long as the human or non-human homolog of the CD91 polypeptide has the ability to bind to the hemopexin-heme complex in the non-human being. In analogy to the above, where the CD91 polypeptide is intended for detection of hemopexin-heme complex in a non-human being, it is generally preferable that the CD91 polypeptide is derived from the same species in which the hemopexin-heme complex is to be detected. Illustrative examples of non-human homologs of CD91 will be familiar to persons skilled in the art, including CD91 from rabbit, mouse, dog, cat, bovine, equine, porcine, sheep, goat or bird origin.

If reference is made to a specific amino acid sequence, identified by a SEQ ID number, or to a specific amino acid region of a specific amino acid sequence, identified by a SEQ ID number, then it is understood that a corresponding amino acid sequence or corresponding amino acid region of a different CD91 homolog or a different CD91 polypeptide homolog is included within the specific amino acid sequence or specific amino acid region, respectively. For example, if reference is made to a fragment within amino acids 2481 to 2942 of SEQ ID NO: 1, then a corresponding amino acid region of a CD91 polypeptide homolog is encompassed. If reference is made to amino acids 2481 to 2942 according to SEQ ID NO: 2, then a corresponding amino acid region of a CD91 polypeptide homolog is encompassed.

The term “fragment of the ligand binding domain III of CD91” or “fragment thereof” or “fragment” or corresponding wording, as used herein, means any fragment of the ligand binding domain III of CD91 which is capable of binding to the hemopexin:heme complex, preferably a human hemopexin-human heme complex. Binding may be with the same affinity as a CD91 polypeptide derived from SEQ ID NO: 1 or as the CD91 polypeptide of SEQ ID NO: 2 or 3 with an affinity that is at least 70%, 80%, 85%, 90%, 95%, 96%, 97%, 98%, 99% or 100% of the affinity, with which the CD91 polypeptide derived from SEQ ID NO: 1 or the CD91 polypeptide of SEQ ID NO: 2 binds to a hemopexin:heme complex, preferably a human hemopexin-human heme complex. Importantly, the fragment does not bind to hemopexin to which a heme is not bound.

The term “CD91 polypeptide having a length of”, as used herein, is to be understood that the CD91 polypeptide is exactly of the indicated length and does not contain additional amino acids resulting in a longer length. However, a CD91 polypeptide defined by its length may comprise additional amino acids at the N- and/or C-terminus which make the polypeptide longer, however, which are not included within the indicated length. Such additional amino acids are from non-CD91 protein sequences, such as a signal peptide and/or a fusion tag, or are CD91 protein partial sequences which are removed from the CD91 polypeptide sequence within the CD91 protein and are a signal sequence, one or more growth factor repeats and/or a transmembrane domain.

As used herein, the term “derived from” means that a CD91 polypeptide has the same amino acid sequence as or is substantially (i.e. at least 90%, 94%, 95%, 96%, 97%, 98% or 99%) identical to the corresponding amino acid sequence of CD91 of the subject or species, from which the CD91 polypeptide is derived. The term “derived from” is also used in association with hemopexin and means that a hemopexin has the same amino acid sequence as or is substantially (i.e. at least 90%, 94%, 95%, 96%, 97%, 98% or 99%) identical to the corresponding amino acid sequence of hemopexin of the subject or species, from which the hemopexin is derived.

The CD91 polypeptide, as referred to herein, may be present in an “at least partially purified”, “isolated” or “purified” form. The term “at least partially purified”, “isolated” or “purified” or the like is used herein to mean that the CD91 polypeptide is provided in an isolated and/or purified form which means that it is separated, isolated or purified from its natural environment or the environment in which it is produced. Thus, the CD91 polypeptide is preferably free or substantially free of material with which it is naturally associated, such as other polypeptides or nucleic acids, or of material with which it is naturally associated during preparation (e.g. material of a cell in cell culture). For example, an at least partially purified CD91 polypeptide may comprise no more than 50% impurities (of total protein), preferably no more than 45%, preferably no more than 40%, preferably no more than 35%, preferably no more than 30%, preferably no more than 25%, preferably no more than 20 impurities (of total protein). Isolated or purified CD91 polypeptide may comprise no more than 15%, preferably no more than 10%, preferably no more than 5% or preferably no more than 1% (of total protein) impurities and is most preferably totally free of impurities. Impurities do not refer to components which are present in the assay liquids for performing the assay.

The CD91 polypeptide may be produced by any method known in the art which is able to produce the CD91 polypeptide, for example the CD91 polypeptide can be produced recombinantly or synthetically, using routine methods and reagents that are well known in the art. Preferably, the CD91 polypeptide is recombinantly produced, i.e. is genetically engineered by genetic recombination, in a suitable host cell (e.g., bacteria, yeast, insect cells, mammalian cells) according to methods known in the art (see, e.g., Ausubel et. al, Curr. Protoc. Mol. Biol., Second Edition, 1992 and Sambrook et al., Molecular Cloning: a Laboratory Manual, 2nd edition, 1989, Cold Spring Harbor Laboratory Press).

Methods of recombinantly producing a polypeptide are well known in the art. For example, a nucleic acid molecule comprising a nucleotide sequence encoding the CD91 polypeptide can be introduced and expressed in suitable host cells (e.g. E. coli), and the expressed CD91 polypeptide can be isolated/purified from the host cells (e.g., in inclusion bodies) using routine methods and readily available reagents. Methods for introducing DNA constructs encoding the CD91 polypeptide into host cells are well known in the art and include, for example, standard transformation and transfection techniques (e.g., electroporation, chemical transformation). A person of ordinary skill in the field of the invention can readily select an appropriate method for introducing a DNA construct into host cells. A variety of methods for expressing proteins in host cells are well known in the art (e.g., IPTG-induced expression in E. coli). A person of ordinary skill in the field of the invention can readily select an appropriate method for expressing the CD91 polypeptide in host cells. The expressed CD91 polypeptide can be isolated from the host cells using known methods and reagents including, e.g., lysozyme treatment, sonication, filtration, salting-out, ultracentrifugation and chromatography. Alternatively, the expressed CD91 polypeptide is secreted into the culture medium by way of a signal peptide and can be recovered from the culture medium by methods known in the art. For example, the CD91 polypeptide can be purified from the host cells or cell lysates of the host cells or from the culture medium by binding to a support using standard techniques and reagents. For example, the CD91 polypeptide is isolated by binding to a solid support via a specific receptor such as an antibody or fragment thereof which is specific for the CD91 polypeptide. Alternatively, the CD91 polypeptide may be isolated by a tag, enabling purification by methods known in the art.

For producing recombinant CD91 polypeptide, a construct comprising a nucleotide sequence encoding the CD91 polypeptide is produced for example by cloning the nucleotide sequence into the cloning site of a vector. Suitable vectors are known in the art and include plasmids, cosmids, artificial chromosomes (e.g. bacterial, yeast or human), bacteriophages, viral vectors (retroviruses, lentiviruses, adenoviruses, adeno-associated viruses) or nano-engineered substances (e.g. ormosils). The vectors preferably comprise one or more suitable sequence(s) capable of effecting the expression in a suitable host. Such a sequence may be a promoter sequence that is recognized by one or more proteins that are endogenous to a host cell and which is capable of directing transcription of a nucleic acid operably linked to the promoter in a host cell. In addition to a promoter sequence, other sequences that can be operably linked to the CD91 polypeptide nucleotide sequence may include, but are not limited to, sequences encoding fusion tags, sequences encoding signal peptides, initiation sequences, terminator sequences, transcriptional and translational stop signals and selectable marker sequences such as an antibiotic resistance gene, e.g., a kanamycin resistance gene. A variety of selectable markers are known in the art and can be used in the present invention. Moreover, the construct may further comprise one or more nucleotide sequences encoding amino acid sequences for desired purposes, for example, protein tags or linker sequences for attaching the protein tag or another kind of tag such as a chemical molecule such as biotin to the CD91 polypeptide. For example, if a protein tag is present, the coding sequence of the protein tag may be inserted into the coding DNA sequence of the CD91 polypeptide, for example behind the start codon or in front of the stop codon while maintaining the reading frame. This creates an N- or C-terminal protein tag on the CD91 polypeptide.

Hemopexin represents the primary line of defense against heme toxicity, attributed at least in part to its ability to bind heme with high affinity (Kd<1 μM) and to function as a heme specific carrier from the bloodstream to the liver. Hemopexin has also been reported to possess serine protease activity and several other functions, such as anti- and pro-inflammatory activities, the ability to inhibit cellular adhesion and binding of certain divalent metal ions (Mauk, M. R., 2011. An alternative view of the proposed alternative activities of hemopexin. Protein Science, Volume 20, pp. 791-805).

Hemopexin is a 61±2 kDa plasma β-1 B-glycoprotein composed of a single 439 amino acids long peptide chain with a carbohydrate content of 20 to 22%, which is formed by two four-bladed β-propeller domains, resembling two thick disks that lock together at a 90° angle and are joined by an interdomain linker peptide. The heme, which is released into the blood as the result of intra- and extra-vascular hemolysis, is bound between the two four-bladed β-propeller domains in a highly hydrophobic pocket formed by the interdomain linker peptide. Residues His213 and His266 coordinate the heme iron atom giving a stable bis-histidyl complex, similar to hemoglobin.

Hemopexin contains 20 to 22% carbohydrates, including sialic acid, mannose, galactose, and glucosamine. Twelve cysteine residues were found in the protein sequence, probably accounting for six disulphide bridges. Hemopexin represents the primary line of defense against heme toxicity thanks to its ability to bind heme with high affinity (K_{d}<1 pM) and to function as a heme specific carrier from the bloodstream to the liver. It binds heme in an equimolar ratio, but there is no evidence that heme is covalently bound to the protein. Further features are: UV extinction coefficient at 280 nm [mL (mg×cm)] of 1.97; theoretical pl of 6.55; and average of hydrophobicity of −0.43 (no high scoring hydrophobic segments).

As used herein, the term “hemopexin” is intended to mean a hemopexin protein as it is naturally found in the blood of a vertebrate such as human, rabbit, mouse, dog, cat, cattle, horse, pig, sheep, goat or bird, preferably human. Preferably, hemopexin is the mature form of human hemopexin consisting of amino acid residues 24 to 462 of the NCBI Reference Sequence NP_000604.1 shown as SEQ ID NO: 4 or an amino acid sequence having at least 60%, 70%, 80%, 85%, 90%, 95%, 96%, 97%, 98%, 99% or 100% identity or at least 50%, 60%, 70%, 80%, 85%, 90%, 95%, or 100% homology to SEQ ID NO: 4.

| SEQ ID NO: 4 | |
|---|---|
| 1 | MARVLGAPVA LGLWSLCWSL AIATPLPPTS AHGNVAEGET KPDPDVTERC SDGWSEDATT |
| 61 | LDDNGTMLFF KGEFVWKSHK WDRELISERW KNFPSPVDAA FRQGHNSVFL IKGDKVWVYP |
| 121 | PEKKEKGYPK LLQDEFPGIP SPLDAAVECH RGECQAEGVL FFQGDREWFW DLATGTMKER |
| 181 | SWPAVGNCSS ALRWLGRYYC FQGNQFLRED PVRGEVPPRY PRDVRDYFMP CPGRGHGHRN |
| 241 | GTGHGNSTHH GPEYMRCSPH LVLSALTSDN HGATYAFSGT HYWRLDTSRD GWHSWPIAHQ |
| 301 | WPQGPSAVDA AFSWEEKLYL VQGTQVYVFL TKGGYTLVSG YPKRLEKEVG TPHGIILDSV |
| 361 | DAAFICPGSS RLHIMAGRRL WWLDLKSGAQ ATWTELPWPH EKVDGALCME KSLGPNSCSA |
| 421 | NGPGLYLIHG PNLYCYSDVE KLNAAKALPQ PQNVTSLLGC TH |

The detection of the hemopexin:heme complex by the CD91 polypeptide can be performed by any method known in the art by which binding of a ligand to a receptor can be detected. Such a method can include an immunoassay, a high performance liquid chromatography (HPLC) assay or a typical ligand-binding assay such as labeled, label-free, thermodynamic or structure-based assay (see Yakimchuk K., Materials and Methods, 2011; 1:199 (last modified: 2020 Mar. 26).

Detection can be performed at a solid phase or in solution. Preferably, the method is a solid phase method. Thereby, the CD91 polypeptide may be linked or suitable to be linked to a solid support. Alternatively, the hemopexin:heme complex is linked to the solid support.

Preferably, the CD91 polypeptide is linked or suitable to be linked to the solid support. Any suitable polymer, preferably synthetic polymer, may be used as solid support. Linkage to the solid support may be direct, which means that the CD91 polypeptide is linked without an intermediate, especially if the affinity of the CD91 polypeptide to the surface of the solid support is high. The linking may thereby be non-specific via adsorption. Examples of surfaces suitable for linking the CD91 polypeptide are charged surfaces to which charged regions of the CD91 polypeptide have a high affinity. Preferably, the surface is a highly charged polystyrene surface, such as present on a MaxiSorp™ plate. According to one embodiment, the solid support may be a resin material or a bead-like particle, both may be suitable for a chromatographic method. Alternatively, linking on the solid support may be indirect which means that the CD91 polypeptide is linked via a linking compound. A linking compound is attached to the solid support as an intermediate compound between the solid support surface and the CD91 polypeptide. Examples of indirect linking are antigen-antibody binding, where an antibody specific to the CD91 polypeptide (which is the antigen) is attached to the solid support and the CD91 polypeptide is linked to the solid support by the antibody. Alternatively, a binding compound for the CD91 polypeptide other than an antibody may be attached to the solid support. Still alternatively, the CD91 polypeptide may comprise a tag by which the CD91 polypeptide is linked to the solid support via an affinity binding partner which is attached to the solid support. Such tags include affinity tags such as chitin binding protein (CBP), maltose binding protein (MBP), Strep-tag, glutathione-S-transferase (GST) or poly(His) tag or a binding partner of a binding pair such as biotin which are known in the art and are discussed herein. Linking of the CD91 polypeptide to the solid support does not interfere with the binding of the hemopexin:heme complex to the CD91 polypeptide.

Preferably, the detection method is an immunoassay, where preferably the presence or concentration of the hemopexin:heme complex in a biological sample is measured through the use of a detection antibody which specifically binds to the hemopexin. Most preferably, the method is an enzyme-linked immunosorbent assay (ELISA) which may be a sandwich ELISA assay.

In an especially preferred embodiment, the amount of the hemopexin:heme complex in the biological sample is determined.

To perform the method, the CD91 polypeptide is preferably linked to a solid support. Then, the biological sample is added to the solid support. A detection antibody is applied over the surface of the solid support so that it can bind to the hemopexin of the hemopexin:heme complex bound be the CD91 polypeptide. The antibody may be linked to an enzyme or may be detected by a secondary antibody that is linked to an enzyme. In the final step, read-out components including the reaction solution containing the substrate of the enzyme are added. The enzymatic reaction produces a detectable signal which may be a color, fluorescent or electrochemical signal. The kind of enzyme and substrate for producing a color, fluorescent or electrochemical signal are known in the art. Between each step, i.e. linking step, adding the biological sample, adding the detection antibody and adding the substrate, the solid support is typically washed with a mild detergent solution to remove any component such as proteins or antibodies that are not or are non-specifically bound. After the final wash step, the solid support is developed by adding an enzymatic substrate to produce the signal. The absorbance, fluorescence or electrochemical signal is measured to determine the presence and/or quantity of the hemopexin:heme complex in the biological sample.

During the experimental investigation of the conditions for detecting the hemopexin:heme complex using a CD91 polypeptide, a classic plate-based format proved to be very effective in detecting and quantitating the hemopexin:heme complex. Thereby, the CD91 polypeptide is attached non-specifically as a coat to a MaxiSorp™ high-binding plate with high affinity for molecules with polar or hydrophilic groups. The hemopexin:heme complex of the sample binds to the CD91 polypeptide attached to the plate and is detected using a rabbit anti-hemopexin antibody and a HRP-conjugated anti-rabbit antibody against the anti-hemopexin antibody. The detection antibody and the secondary antibody are known in the art and are commercially available. For example, the detection antibody is an anti-hemopexin antibody such as a rabbit polyclonal antibody from Abcam (Ab48135). The secondary antibody is an anti-detection antibody such as an unspecific goat anti-rabbit antibody such as from Seracare (5220-0336) which is linked to the enzyme horseradish peroxidase (HRP), which catalyzes the oxidation of various organic substrates by hydrogen peroxide. As the peroxidase substrate, TMB (3,3′,5,5′-tetramethylbenzidine) is used to generate the detectable signal. Thereby, the reaction between the TMB substrate and HRP produces a measurable color change (intense blue color that can be read directly at 650 nm or a deep yellow color that can be read at 450 nm after stopping with an acid solution) that correlates with the level of the hemopexin:heme complex in the sample. In this way, the amount of the hemopexin:heme complex can be determined. In general, the conditions as described in Example 2 below can be used.

For the detection of the hemopexin:heme complex, a detection antibody to the hemopexin in the hemopexin:heme complex may be used. A “detection antibody” is an antibody which is specific for the hemopexin and binds to the hemopexin in the hemopexin:heme complex for the detection thereof. The detection antibody does not interfere with the binding of the hemopexin to the CD91 polypeptide and to the heme. The skilled person knows how to prepare antibodies to a specific protein. Moreover, antibodies specific to hemopexin which can be used in the present invention are commercially available, such as the rabbit polyclonal anti-hemopexin antibody from Abcam (Ab48135).

The detection of the hemopexin:heme complex in the biological sample via the detection antibody may occur via a “secondary antibody” which is able to bind to the detection antibody, but not any antigen that is present in the reaction solution. The secondary antibody is usually an unspecific antibody which binds to the heavy chain of the detection antibody, thereby differentiating between antibodies of different species, and does not interfere with the binding of the detection antibody binding to hemopexin. Thus, detection antibodies are usually derived from a kind of species such as rabbit species and the secondary antibody is directed to species specific sequences of the detection antibody. Secondary antibodies are commercially available such as from Seracare (5220-0336).

The term “specific binding”, “specific binding of an antibody”, “specific antibody” or a corresponding term refers to a binding reaction wherein a compound or antibody binds to a particular binding partner which is an antigen in the case of an antibody, whereas it does not bind in a substantial amount (less than 10% of the binding to the particular protein) to other molecules present in the reaction mixture. Generally, a binding partner such as an antibody of a binding pair that “specifically binds” to its corresponding binding partner such as an antigen in the case of antibody has an equilibrium affinity constant greater than about 10⁵ (e.g., 10⁶, 10⁷, 10⁸, 10⁹, 10¹⁰, 10¹¹, 10¹² or more) mole/liter for that target molecule.

The term “antibody”, as used herein, includes a full-length antibody, as known in the art, or an “antibody derivative”, defined herein as a molecule comprising at least one antibody variable domain, but not comprising the overall structure of an antibody. The antibody derivative is still capable of binding a target molecule. A derivative may be antibody fragment such as Fab, Fab2, scFv, Fv, or parts thereof, or other derivatives or combinations of immunoglobulins such as nanobodies, diabodies, minibodies, camelid single domain antibodies, single domains or Fab fragments, domains of the heavy and light chains of the variable region (such as Fd, VL, including Vlambda and Vkappa, VH, VHH) as well as mini-domains consisting of two beta-strands of an immunoglobulin domain connected by at least two structural loops.

Linking of the CD91 polypeptide or detection of the hemopexin:heme complex may also be made by an “antibody mimetic”, which is defined herein as an organic compound that, like an antibody, can specifically bind the CD91 polypeptide or the hemopexin:heme complex, but is not structurally related to antibodies. Antibody mimetics are usually artificial peptides or proteins with a molar mass of about 3 to 20 kDa. Non-limiting examples of antibody mimetics are affibodies, affilins, affimers, affitins, anticalins, avimers, DARPins, fynomers, Kunitz domain peptides, monobodies, Z domain of Protein A, Gamma B crystalline, ubiquitin, cystatin, Sac7D from Sulfolobus acidocaldarius, lipocalin, A domain of a membrane receptor, ankyrin repeat motive, SH3 domain of Fyn, Kunits domain of protease inhibitors, the 10^{th} type III domain of fibronectin, synthetic heterobivalent or heteromultivalent ligands (Josan et. al, Bioconjug. Chem., 2011; 22:1270-8, Shallal et. al, Bioconjug. Chem., 2014; 25:393-405 and Xu et. al, PNAS, 2012; 109:21295-300).

Linking of the CD91 polypeptide or detection of the hemopexin:heme complex may also be made by any other compound which specifically binds to the CD91 polypeptide or hemopexin, respectively, without interfering with the binding of other components which are necessary for the detection of the hemopexin:heme complex in the biological sample. “Specific binding” is meant in the above sense, namely not binding in a substantial amount (less than 10% of the binding to the CD91 polypeptide or hemopexin:heme complex, respectively) to other molecules present in the reaction mixture.

The detection method of the present invention may also be performed by linking the hemopexin:heme complex of a biological sample to a solid support via a specific compound such as an antibody, then adding the CD91 polypeptide and detecting the hemopexin:heme complex via the detection of the bound CD91 polypeptide.

The detection of the hemopexin:heme complex in the biological sample may also occur in solution, which may be the biological sample comprising the hemopexin:heme complex or a mixture of the biological sample and a solution comprising components such as buffer or salt facilitating the completion of the detection method. Thereby, the binding of the CD91 polypeptide to the hemopexin:heme complex is made in solution. For detection of the complex of the CD91 polypeptide and hemopexin:heme complex, in analogy to the detection using a solid phase, detection antibody is added. The whole complex is then linked to a solid support where the read-out is carried out.

The biological sample can be any fluid, in which a hemopexin:heme complex, may be present.

The biological sample may be any biological fluid from a subject, in which a hemopexin:heme complex, is present or is expected to be present. The hemopexin may be endogenous hemopexin and/or may be exogenous hemopexin which is administered to the subject, preferably in order to treat hemolysis or a condition associated with hemolysis. Examples of a biological sample may be blood, cerebrospinal fluid (CSF), amniotic fluid, urine, saliva, mucus, sweat, bronchoalveolar lavage, sputum, semen, tears, fecis and/or bile. The blood may be unprocessed (and remain whole blood) or may be processed blood. Preferably, the blood used in the use and method of the present invention is processed blood. Processed blood may include plasma or serum.

The biological sample may be derived from a patient having a condition associated with hemolysis. Preferably, the condition associated with hemolysis is an acute hemolytic condition and/or a chronic hemolytic condition, more preferably selected from the group consisting of sickle cell anemia, hemolytic anemia, aplastic crisis, hyper-hemolytic crisis, transfusion-induced hemolysis, hemolytic uremic syndrome, myocardial infarct, acute chest syndrome, pulmonary hypertension, leg ulcer, growth retardation, bone infarcts, pre-eclampsia, renal failure, acute kidney injury, acute respiratory distress syndrome (ARDS), stroke including hemorrhagic stroke, subarachnoid hemorrhage, intra-cranial hemorrhage (ICH), splenic sequestration, splenic infarcts, an autoimmune disease including autoimmune hemolytic anemia, microbial infection, increased susceptibility to infection, malaria infection, trauma, a transplant related condition, open heart surgery using cardiopulmonary bypass, burn such as hemoglobinemia or hemoglobinuria accompanied with hemolysis after a burn, hereditary spherocytosis, hereditary elliptocytosis, thalassemia, congenital dyserythropoietic anemia, paroxysmal nocturnal haemoglobinuria (PNH), systemic lupus erythematosus and chronic lymphocytic leukemia. Further preferred is that the condition associated with hemolysis is sickle cell anemia.

The biological sample can be any fluid resulting from a manufacturing process, in which a hemopexin:heme complex, may be present. Examples of such fluids may comprise an intermediate or final product of a protein manufacturing process. Such a process may yield a human plasma derived protein or a human protein recombinantly expressed. For example, the term “biological sample” also includes cell culture supernatants and mixtures of cell culture supernatants with other components such as heme.

In a preferred embodiment of the invention, the CD91 polypeptide used may further comprise a signal peptide and/or one or more tags.

The CD91 polypeptide may have added additional components or additional components may be added which are useful for isolating the CD91 polypeptide and/or for carrying out the method of the present invention. The additional components do not participate in the binding to the hemopexin-heme complex. If they are derived from a CD91 protein, they are not immediately adjacent to the CD91 polypeptide sequence within the CD91 protein and do not participate in the binding to the hemopexin-heme complex. Suitable additional components may be polypeptide sequences such as a signal peptide which is a short peptide sequence (usually 16-30 amino acids long) present at the N-terminus of a newly synthesized CD91 polypeptide and is used to direct the CD91 polypeptide into the secretory pathway. Signal peptides for use in the present invention are known in the art. Preferably, the signal peptide linked to the CD91 polypeptide is derived from the same CD91 protein. The signal peptide is cleaved from the CD91 polypeptide during secretion and is not part of the CD91 polypeptide used in the assay of the present invention.

Other additional components may be one or more tags. A tag is a component fused or attached to the CD91 polypeptide, in order to provide desired properties for the CD91 polypeptide. The tag may be a protein tag. As it is fused to the CD91 polypeptide usually at the N- and/or C-terminus, it is also called a fusion tag. Fusion tags are affinity tags allowing protein purification via affinity to a binding partner. Purification occurs via binding to the binding partner by methods known in the art, such as chromatographic methods. Affinity partners, methods for purification via the affinity partners and systems and kits are known in the art and/or commercially available (see e.g. Kimple et. al, Curr Protoc Protein Sci., 2013; 73:9.9.1-9.9.23). Tags may also be useful for detection such as via the binding partner, fluorescence or the presence of recognition motifs, for increasing expression, for increasing solubility, for improving protein folding or for easier separation for example in HPLC due to increased polarity. Example of fusion tags are known in the art and include affinity tags allowing isolation via an affinity technique. Examples are chitin binding protein (CBP), maltose binding protein (MBP), Strep-tag, glutathione-S-transferase (GST) or poly(His) tag, allowing isolation is via an affinity technique (affinity tags). For example, the poly(His) tag binds to metal matrices. Solubilization tags are used especially for recombinant proteins expressed in chaperone-deficient species such as E. coli to assist in the proper folding in proteins and keep them from precipitating. Examples are thioredoxin and poly(NANP). Some affinity tags have a dual role as a solubilization agent, such as MBP and GST. Chromatography tags are used to alter chromatographic properties of the protein to afford different resolution across a particular separation technique. Examples are polyanionic amino acids, such as FLAG-tag. Epitope tags are short peptide sequences with high-affinity to antibodies. Examples are V5-tag, Myc-tag, HA-tag, Spot-tag, T7-tag and NE-tag. These tags are particularly useful for western blotting, immunofluorescence and immunoprecipitation experiments. Fluorescence tags allow visual readout. An example is GFP and its variants are the most commonly used fluorescence tags. More advanced applications of GFP include using it as a folding reporter (fluorescent if folded, colorless if not). Alternatively, a tag may be an enzyme allowing detection of the CD91 polypeptide such as horseradish peroxidase which allows chemiluminescent detection. Alternatively, a tag may be a binding partner of a binding pair allowing detection of the CD91 polypeptide via binding of the tag to its binding partner. An example is the biotin and avidin/streptavidin system, whereby, as the tag, biotin may be bound to the CD91 polypeptide and avidin/streptavidin is bound to the solid support for capturing the CD91 polypeptide. The tags may be removable by chemical or enzymatic agents, thus allowing the CD91 polypeptide to be separated from the tag, for example, after binding of the fusion tag to its affinity partner during isolation.

In a preferred embodiment of the present invention, the CD91 polypeptide is linked to a solid support, preferably a solid support with a highly charged polystyrene surface, more preferably a MaxiSorp™ plate.

A solid support as used herein is any solid support which allows the binding of the CD91 polypeptide or of the hemopexin:heme complex of the biological sample for the detection of the hemopexin:heme complex. Preferably, the CD91 polypeptide is linked to the solid support. Alternatively, a solid support is any solid support which allows the binding of the CD91 polypeptide via an attached tag via the affinity partner of the tag which has already been attached to the solid support for isolation, purification or detection. Examples of a solid support are a plate, preferably a well plate, a bead, a magnetic bead, a resin, a column, whereby beads or resin may be part of the column or may constitute the column, a filter or a membrane. A plate-based method is preferably applied in linking the CD91 polypeptide for the detection of the hemopexin:heme complex, as it may have the advantage of higher throughput, greater sensitivity and greater ease of implementation over non plate-based methods. The plate is more preferably a well plate, more preferably a multi-well plate such as a 6, 12, 24, 48 or 96 well plate. Still more preferably, the surface of the plate is a highly charged polystyrene surface, such as present on a MaxiSorp™ plate.

The detection of a hemopexin:heme complex in a biological sample by a CD91 polypeptide may serve to monitor hemolysis in a subject or to monitor treatment of hemolysis or a condition associated with hemolysis, preferably to monitor treatment of hemolysis or a condition associated with hemolysis with hemopexin.

Heme complexation by hemopexin directly protects cells against heme toxicity. In a subject suffering from hemolysis, the expression of hemopexin is increased to complex and detoxify toxic heme. The hemopexin:heme complex is then cleared from the body. However, as endogenous hemopexin is produced to a limited extent in a body even if the subject suffers from hemolysis, the produced hemopexin may not be sufficient to complex abundant heme. Therefore, an additional treatment of hemolysis will become necessary. Such treatment may be the administration of exogenous hemopexin for complexing and detoxifying abundant heme.

Hemopexin serves to complex and detoxify heme in biological fluids. Rising heme levels during hemolysis result in increased, however limited, expression of hemopexin. Therefore, endogenous hemopexin:heme complex levels are indicative of hemolysis or the severity of hemolysis. Thereby, the higher the hemopexin:heme complex levels are, the more severe is the hemolysis. However, this correlation is limited, as hemopexin is produced to a limited extent only. Therefore, detecting the concentration of hemopexin:heme complex in a body fluid is indicative of the occurrence of hemolysis and the severity of hemolysis in a subject.

Additionally, detection and quantification of hemopexin:heme complex in a subject may be useful for monitoring whether treatment of hemolysis in a subject is effective. Thereby, if levels of hemopexin:heme complex in a subject with hemolysis decrease with treatment, this may be indicative of an effective treatment of hemolysis. Thus, the determination of the level of the hemopexin:heme complex in a subject may serve for monitoring the effectiveness of a hemolysis treatment, allowing the adaptation of the treatment dependent on the amount of the hemopexin:heme complex.

Moreover, detection and quantification of hemopexin:heme complex in a subject may be useful for monitoring whether treatment of hemolysis in a subject with hemopexin is effective. The administration of exogenous hemopexin may result in a rise of hemopexin:heme complex levels above endogenous hemopexin:heme complex levels, if high heme levels are produced during hemolysis and endogenous hemopexin is not sufficiently produced to complex the heme completely. Such high hemopexin:heme complex levels, therefore, indicate efficient complexation of abundant heme by the exogenous hemopexin, but may also indicate that a high level of heme is produced in the subject, indicating that hemolysis is still going on and is in a severe state. Thus, the determination of the level of the hemopexin:heme complex in a subject may serve for monitoring the effectiveness of a hemopexin treatment, allowing the adaptation of the hemopexin treatment dependent on the level of the hemopexin:heme complex in the subject.

Alternatively, detection of hemopexin:heme complex may serve to monitor the efficacy of the use of hemopexin as a pharmaceutical in subjects with hemolysis during clinical trials with hemopexin. Quantification of hemopexin:heme complex levels above certain levels may be indicative that the therapeutically administered hemopexin is effective in complexing heme and is, thus, effective as a pharmaceutical.

Consequently, in a preferred embodiment, the use of the invention is for diagnosing or monitoring hemolysis in a subject, or for monitoring a treatment of hemolysis or a condition associated with hemolysis in a subject.

In a further aspect, the invention relates to an in vitro method for detecting a hemopexin:heme complex in a biological sample, comprising incubating the biological sample with a CD91 polypeptide as defined above and detecting the binding between the CD91 polypeptide and the hemopexin:heme complex.

All embodiments defined above also apply to this method of the invention.

According to one embodiment, the in vitro method for detecting a hemopexin:heme complex in a biological sample comprises at least the following method steps:
incubating the biological sample with a CD91 polypeptide as defined above and
detecting the binding between the CD91 polypeptide and the hemopexin:heme complex.

According to one embodiment, the in vitro method for detecting a hemopexin:heme complex in a biological sample comprises at least the following method steps:
providing a solid support to which the CD91 polypeptide as defined above is linked,
incubating the biological sample with the CD91 polypeptide,
optionally, performing a washing step,
detecting the binding between the CD91 polypeptide and the hemopexin:heme complex, and
determine the amount of the hemopexin:heme complex in the biological sample.

In a further preferred embodiment, the method of the invention is used to determine the amount of heme in a biological sample, preferably blood or serum, which is accessible to the binding to hemopexin.

It is known that not all heme present in the serum is accessible to hemopexin, such that bound to hemoglobin. The method of the present invention can also be used to determine the amount of accessible heme in a biological sample, i.e. to determine the amount of heme which is accessible to form a complex with hemopexin. For this purpose, excess hemopexin is added to the biological sample, which creates additional hemopexin:heme complex. The amount of the hemopexin:heme complex is then determined in samples with and without excess hemopexin added. The difference in molar concentration between the two samples is the concentration of hemopexin-accessible heme (as hemopexin:heme bind in a 1:1 ratio).

According to an embodiment, a method to determine the amount of accessible heme in a biological sample comprises at least the following steps:
addition of excess hemopexin to the biological sample, which creates additional hemopexin:heme complex (spiked) and provide a biological sample without excess hemopexin added (un-spiked),
determine the amount of the hemopexin:heme complex in biological samples with (spiked) and without excess hemopexin added (un-spiked) by applying, preferably, an in vitro method for detecting a hemopexin:heme complex in a biological sample as described herein,
calculate the difference in molar concentration of the determined hemopexin:heme complex between the two samples, with (spiked) and without excess hemopexin added (unspiked), in order to obtain the concentration of hemopexin-accessible heme based on a premise of hemopexin:heme binding in a 1:1 ratio.

According to an embodiment, a method to determine the relative accessible heme concentration (C) in a biological sample comprises at least the following steps:
addition of excess hemopexin to the biological sample, which creates additional hemopexin:heme complex (spiked) and provide a biological sample without excess hemopexin added (un-spiked),
determine the concentration (B) of the hemopexin:heme complex in biological samples with (spiked) and the concentration (A) without excess hemopexin added (un-spiked) by applying, preferably, an in vitro method for detecting a hemopexin:heme complex in a biological sample as described herein,
determine the concentration of hemopexin-accessible heme by subtracting the concentration of hemopexin:heme complex present in the un-spiked samples (A) from hemopexin spiked samples (B), whereby the resulting delta hemopexin:heme complex concentration (B-A) is divided by the molecular weight of hemopexin given that heme binds to hemopexin at a 1:1 ratio,
obtaining the relative concentration (C) of the hemopexin-accessible heme within a sample by dividing the delta value of the previous step by the molecular weight of hemopexin.

Thus, the relative accessible heme concentration may be calculated as (C)=([spiked (B)−unspiked (A)]/63000)*1000 (μM).

The invention also relates to a method for separating in vitro or for extracorporeal separating a hemopexin:heme complex from a biological sample, comprising incubating the CD91 polypeptide as defined above with the biological sample, and separating the CD91 polypeptide-hemopexin:heme complex from the biological sample.

Separation of a hemopexin:heme complex from a biological sample means that the hemopexin:heme complex is removed or depleted from the biological sample by attaching it to a CD91 polypeptide and removal of the resulting complex of CD91 polypeptide and hemopexin:heme complex from the biological sample. The separation of the hemopexin:heme complex from the biological sample may occur by any method known to the skilled person. Principally, the separation corresponds to the first part of the procedure of detection of the hemopexin:heme complex of the present invention, where the CD91 polypeptide and the hemopexin:heme complex are attached together. Thus, the above description applies to the aspect of the separation of the hemopexin:heme complex from the biological sample. Preferably, as described above, the CD91 polypeptide is linked to a solid support, the solid support having linked the CD91 polypeptide is contacted with the biological sample and the solid support having linked the CD91 polypeptide and the attached hemopexin:heme complex is removed from the biological sample. Thereby, effective removal may be obtained if the biological sample is conducted, for example through a column, over a resin, a filter or a membrane or contacted with beads which may then be removed, for example by centrifugation or magnetic separation.

Separation of the hemopexin:heme complex from a biological sample may be in vitro or may be extracorporeal. It may serve to clear the biological sample from the hemopexin:heme complex. “Extracorporeal” separation means a procedure which is performed outside the subject with a blood sample obtained from the subject. Thereby, blood is taken from a subject, optionally processed as indicated above, and contacted with the CD91 polypeptide. The resulting complex of CD91 polypeptide and hemopexin:heme complex is separated from the blood and the cleared blood is returned to the subject. The extracorporeal procedure may be performed in vitro, i.e. blood is collected from a subject, contacted with the CD91 polypeptide in vitro, the resulting complex of CD91 polypeptide and hemopexin:heme complex is separated and the blood is returned to the body. The extracorporeal procedure may alternatively be performed in a circuit procedure, where blood circulates from the subject to the subject with separation of the hemopexin:heme complex, before it is returned to the circulation of the subject. All of the procedures where the blood is carried outside the body are made in an extracorporeal circuit. Preferably, the procedure is apheresis in which the blood of a subject is passed through an apparatus that separates out the hemopexin:heme complex by using the CD91 polypeptide and returns the remainder to the circulation.

A further aspect of the present invention is the CD91 polypeptide as defined above for use in a method of treating a condition, provided that for said condition it is desirable to block or reduce the interaction of the hemopexin:heme complex to its endogenous CD91 receptor. In such a situation, the polypeptide of the invention may be administered to a subject in need thereof as a decoy for the hemopexin:heme complex in order to block or decrease CD91 receptor-mediated endocytosis and/or further downstream signaling.

Preferably, the condition associated with hemolysis is an acute hemolytic condition and/or a chronic hemolytic condition, more preferably selected from the group consisting of sickle cell anemia, hemolytic anemia, aplastic crisis, hyper-hemolytic crisis, transfusion-induced hemolysis, hemolytic uremic syndrome, myocardial infarct, acute chest syndrome, pulmonary hypertension, leg ulcer, growth retardation, bone infarcts, pre-eclampsia, renal failure, acute kidney injury, acute respiratory distress syndrome (ARDS), stroke including hemorrhagic stroke, subarachnoid hemorrhage, intra-cranial hemorrhage (ICH), splenic sequestration, splenic infarcts, an autoimmune disease including autoimmune hemolytic anemia, microbial infection, increased susceptibility to infection, malaria infection, trauma, a transplant related condition, open heart surgery using cardiopulmonary bypass, burn such as hemoglobinemia or hemoglobinuria accompanied with hemolysis after a burn, hereditary spherocytosis, hereditary elliptocytosis, thalassemia, congenital dyserythropoietic anemia, paroxysmal nocturnal haemoglobinuria (PNH), systemic lupus erythematosus and chronic lymphocytic leukemia.

The term “condition associated with hemolysis” refers to any condition in a subject, where hemolysis occurs including a condition where hemolysis is the cause of the condition or a condition which causes hemolysis.

Treatment of hemolysis or a condition associated with hemolysis involves complexing the hemopexin:heme complex in the blood of a subject. Therefore, the CD91 polypeptide may be administered to the subject to complex the hemopexin:heme complex in a body fluid.

For the use of the CD91 polypeptide in a treatment, the CD91 polypeptide is provided as a pharmaceutical composition comprising the CD91 polypeptide and a pharmaceutically acceptable carrier. For the production of the pharmaceutical composition, the CD91 polypeptide has to be in a pharmaceutical dosage form comprising the CD91 polypeptide and a mixture of ingredients such as pharmaceutically acceptable carriers which provide desirable characteristics. The pharmaceutical composition comprises one or more suitable pharmaceutically acceptable carrier(s) which is/are known to those skilled in the art.

The pharmaceutical composition can be formulated for systemic, nasal, parenteral, enteral or topic administration. Parental administration includes subcutaneous, intracutaneous, intramuscular, intravenous or intraperitoneal administration.

The pharmaceutical composition can be formulated as various dosage forms including solid dosage forms for oral administration such as capsules, tablets, pills, powders and granules, liquid dosage forms for oral administration such as pharmaceutically acceptable emulsions, microemulsions, solutions, suspensions, syrups and elixirs, injectable preparations, for example, sterile injectable aqueous or oleaginous suspensions and dosage forms for topical or transdermal administration.

The specific therapeutically effective dose level for any particular subject will depend upon a variety of factors including the dosage form, the age, body weight and sex of the subject, the duration of the treatment and like factors well known in the medical arts.

In a preferred embodiment, the method for treating hemolysis or a condition associated with hemolysis comprises the use of the CD91 polypeptide for separating a hemopexin:heme complex from blood. All embodiments described above in the context of separating the hemopexin:heme complex from the blood also apply to this embodiment.

Especially, the separating may be extracorporeal, such as by apheresis, or the separating may be in vivo in the blood.

For removing the complex of the CD91 polypeptide-hemopexin:heme complex from the body fluid, the complex is cleared by the subject and, thus, removed from the subject by natural processes. Alternatively, the complex of the CD91 polypeptide-hemopexin:heme complex is separated extracorporeal either in vitro or in an extracorporeal circuit from the blood of the subject. Thereby, the blood is removed from the subject and the complex of the CD91 polypeptide-hemopexin:heme complex is separated from the blood which is then returned to the subject. The separation may be via binding the complex of the CD91 polypeptide-hemopexin:heme complex to a solid support via the CD91 polypeptide or the hemopexin:heme complex and removing the solid support from the blood. Binding of the CD91 polypeptide or the hemopexin:heme complex to a solid support is described herein.

The present invention also discloses a method of treating a subject by administering a pharmaceutically effective amount of the CD91 polypeptide. All embodiments described above also apply to this method of the invention.

The subjects which are treated by the CD91 polypeptide are animals such as vertebrates such as human, rabbit, mouse, dog, cat, cattle, horse, pig, sheep, goat or bird, preferably human.

A further aspect of the present invention refers to a kit comprising the CD91 polypeptide. A kit is an assembly of separate units comprising the CD91 polypeptide and further ingredients which are necessary carrying out the method of the present invention. The CD91 polypeptide may be present in free form in solution or in solid form such as a powder for reconstitution in a solution or may be linked to a solid support, as defined herein. If not already linked to a solid support, a solid support may be additionally present in the kit to which the CD91 polypeptide is linked during the conduct of the method. The CD91 polypeptide may be either covalently or non-covalently linked to the support. The CD91 polypeptide may be either directly or indirectly linked to the support. The CD91 polypeptide may be provided with a bound tag, as described herein. Optionally, further reagents or solutions may be present. Such a solution may be a solution adapted for linking the CD91 polypeptide to the solid support, a solution adapted for binding the hemopexin:heme complex in the biological sample to the CD91 polypeptide, a solution including the anti-hemopexin detection antibody adapted for binding the anti-hemopexin detection antibody to the hemopexin:heme complex, a solution the secondary antibody adapted for binding the secondary antibody to the detection antibody and/or a solution including the substrate adapted for the enzymatic reaction of the substrate by the enzyme linked to the secondary antibody. Instead of an adapted solution, the anti-hemopexin detection antibody, the secondary antibody or the substrate may also be present in solid form such as a powder or may be present in a storage solution. The solid form or the storage solution is for use in the solutions for carrying out the respective reactions. The kit may also contain instructions for carrying out the method of the present invention.

A further aspect of the present invention relates to a solid support having linked the CD91 polypeptide. The solid support is as defined herein. The CD91 polypeptide may have bound a tag, as defined herein.

A summary of the sequences referred to herein is set out in the following Table:

| SEQ | |
|---|---|
| ID NO: | Description |
| 1 | Amino acid sequence of human CD91/LRP1 protein |
| 2 | Amino acid sequence of ligand binding domain III of human |
| | CD91 |
| 3 | Amino acid sequence of a preferred embodiment of the CD91 |
| | polypeptide |
| 4 | Amino acid sequence of human hemopexin |
| 5 | Linker |
| 6 | Flag tag |

The invention is now further described by the following examples and figures, which are intended to illustrate, but not to limit the invention.

### FIGURE LEGENDS

FIG. 1. Example workflow for kinetic characterization of the interaction between a His-tagged protein and a target analyte

The assay consists of 5 assay steps. Step 1: equilibration, Step 2: loading (capture) of His-tagged protein, Step 3: baseline, Step 4: analyte association, Step 5: analyte dissociation. For further details see TechNote 43 (6).

FIG. 2. Schematic representation of huLRP1 soluble minireceptors (5)

Each of the four soluble minireceptors is depicted in comparison to the full-length endogenous huLRP1. The four putative ligand-binding domains are labelled with numerals I, II, III and IV.

FIG. 3. Expression of huLRP1 minireceptors in ExpiCHO-S™ cells and purification.
(A) Coomassie-stained reducing SDS-PAGE of huLRP1 minireceptors encoding binding domains I-IV (indicated above the gel) produced in transiently transfected ExpiCHO-S™ cells. With co-expression of huLRPAP1 (RAP) (+) there is a relative increase in expression levels.
(B) Anti-His western blot of reducing SDS-PAGE of huLRP1 minireceptors encoding binding domains I-IV (indicated above the gel) produced in transiently transfected ExpiCHO-S™ cells.
(C) Coomassie-stained reducing SDS-PAGE of huLRP1 minireceptors encoding binding domains I-IV (indicated above the gel) purified by tandem Nickel and size exclusion chromatography.

FIG. 4. Representative sensograms for each of the LRP1 domains analysed regarding its ability to bind heme-hx complexes.

Individual LRP1 domains were immobilized on the biosensor surface. After baseline recording four concentration of heme-hpx complexes were tested (2.5, 1.25, 0.625 and 0.312 μM). For LRP1.3 the reference subtracted and processed kinetic dataset was globally fitted using a 1:1 binding model. The fitting accuracy was described by Chi² and R² shown in Table 5. Heme-hpx is shown in red curves, fitted curve as solid black line. LRP1.1, LRP1.2, LRP1.4 and LRP1.5 were not fitted.

FIG. 5. Representative sensograms derived from manual runs for all four LRP1 domains.

Polyclonal anti hemopexin immobilised on CM5 with subsequent heme-Hpx capturing step. (A) LRP1 domain 3 and (B) domain 2 used as analyte at 10 μM. (C) Instead of heme-Hpx, unbound Hpx was captured with LRP1 domain 3 as analyte (10 μM) as negative control. All sensograms are reference subtracted.

FIG. 6. Qualitative Hemopexin capture assay.
(A) Schematic illustration of the assay setup (B) Representative sensograms of LRP1.3 binding to heme-hx complex, demonstrating dose dependent binding.

FIG. 7. SPR sensograms of the interactions between heme-hpx and LRP1.3
(A) Schematic illustration of the assay setup. Representative sensograms of (B) heme-hpx complex and (C) lack of binding of hemopexin to LRP1.3. A steady state 1:1 fit of (D) heme-hx complex binding to LRP1.3 and the respective Scatchard plot (E) showing slight negative cooperative interaction.

FIG. 8. Representative sensorgrams and the respective steady state fits of heme-hpx complexes of different batches
(A) Hpx Batch: TO411122 (B) Hpx Batch: TO341022BM (“New”) and (C) Hpx Batch: TO342022B (“Aged”) binding to LRP1.3

FIG. 9. The hemopexin:heme assay is specific for hemopexin bound to heme (Hemopexin:heme) and does not bind free hemopexin (Hemopexin). A 7-point standard curve was prepared using either Hemopexin:heme complex or free hemopexin.

### EXAMPLES

### Example 1

### 1. Introduction

Hemopexin (HPX) is a plasma glycoprotein that binds to free heme with high affinity. Heme released from heme-binding proteins due to internal hemorrhage, hemolysis, myolysis, or other cell damage is highly toxic due to oxidative and pro-inflammatory effects. Hemopexin scavenges free heme, and this heme-hemopexin (heme-hpx) is taken up by liver by CD91/LRP1 (low density lipoprotein receptor-related protein 1) receptor-mediated endocytosis. Plasma hemopexin helps in the metabolic processing of heme and inhibiting the toxicity resulting from the oxidative catalytic activity of heme.

CD91, also known as low density lipoprotein receptor-related protein 1 (LRP1, synonyms alpha-2-macroglobulin receptor, apolipoprotein E receptor), was first described by Herz et. al, The EMBO Journal, 1988; 7(13):4119-27. The human protein is a ˜600 kDa precursor protein which is proteolytically processed by furin to a 515 kDa alpha-chain and a 85 kDa beta-chain, which are non-covalently associated. It is expressed on a wide spectrum of cell types including macrophages, hepatocytes, fibroblasts, adipocytes, neurons, vascular smooth muscle cells and syncytiotrophoblasts, most abundantly in vascular smooth muscle cells, hepatocytes and neurons. CD91 orthologs are found in vertebrates, where the nucleotide and amino acid structures have been elucidated for a large number of species.

Analysis of the biogenesis and ligand-binding properties of LRP1 is hampered by the difficulties in recombinant expression of the full-length receptor. Based on previous studies (Bu et. al, J Biol Chem., 1996; 271(36):22218-24; Neels et. al, J Biol Chem., 1999; 274(44):31305-11 and Willnow et. al, J Biol Chem. 1994; 269(22):15827-32) the four recombinant soluble ligand binding domains of LRP1 (I-IV) were generated and used for testing its binding to heme-hx complex.

### 2. Study Objectives

First objective was the investigation and ability to express and purify recombinant versions of the four clusters of ligand-binding repeats contained within CD91/LRP1

Second objective was to investigate, which of the four domains is able to bind heme-hpx complexes on two different platforms (BLI and SPR)

### 3. Materials and Methods

3.1 Cell Culture

ExpiCHO-S™ cells and the mammalian expression vector pcDNA3.1 were obtained from Invitrogen™, Thermo Fisher Scientific (R790-07, V790-20). Cells were cultured in GIBCO@ExpiCHO Expression Medium (Invitrogen™, Thermo Fisher Scientific). Cells were maintained at 37° C. in incubators with an atmosphere of 8% CO2.

3.2 Generation of cDNA Expression Plasmids

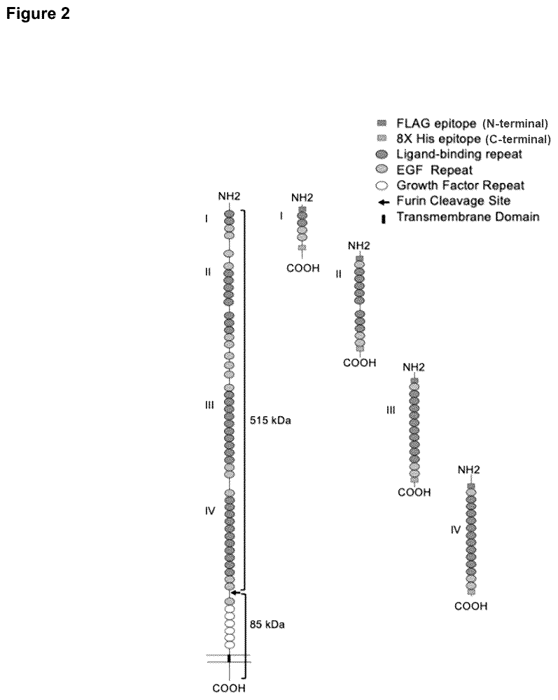

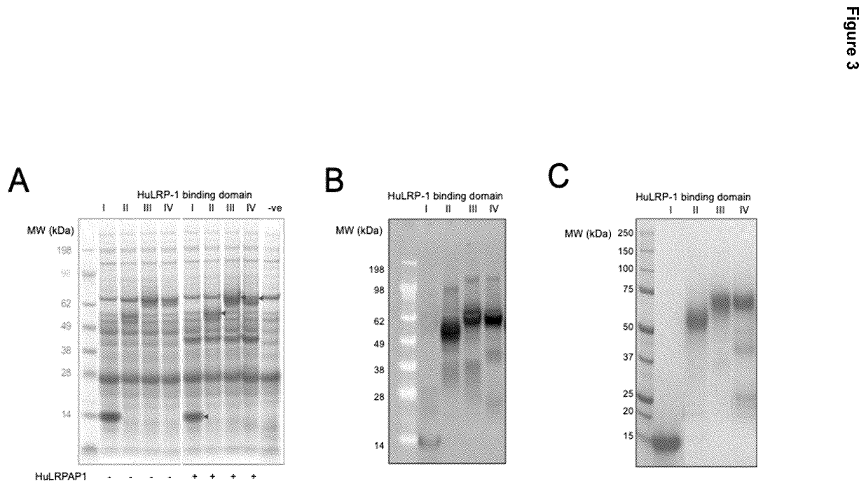

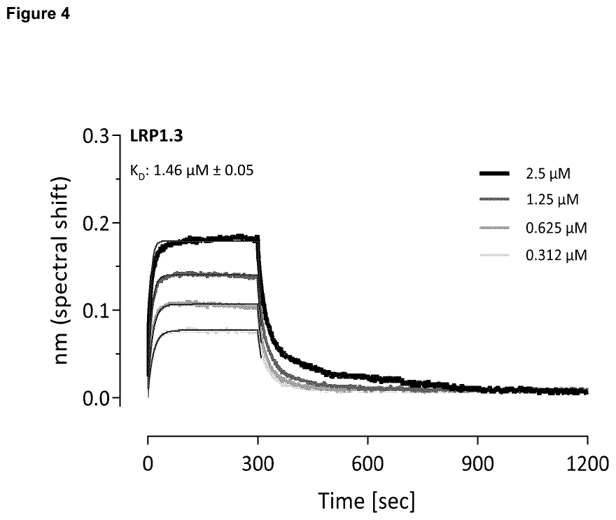

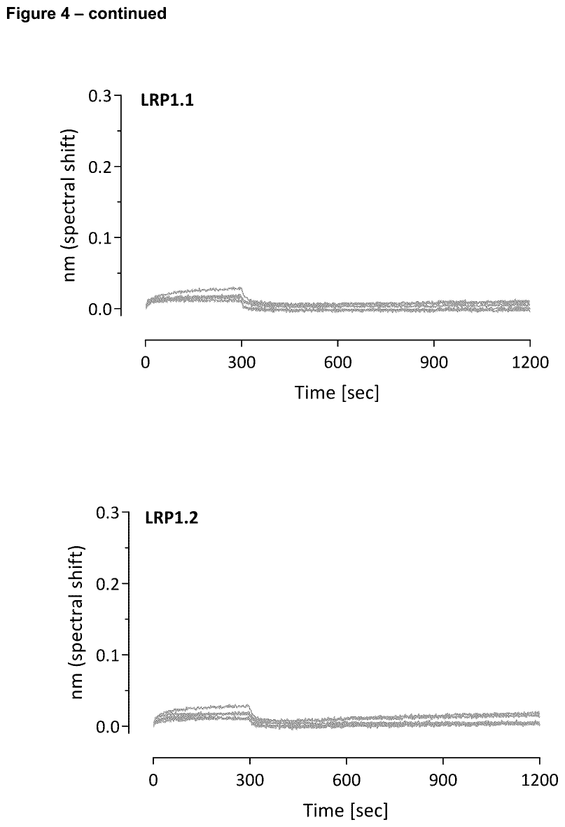

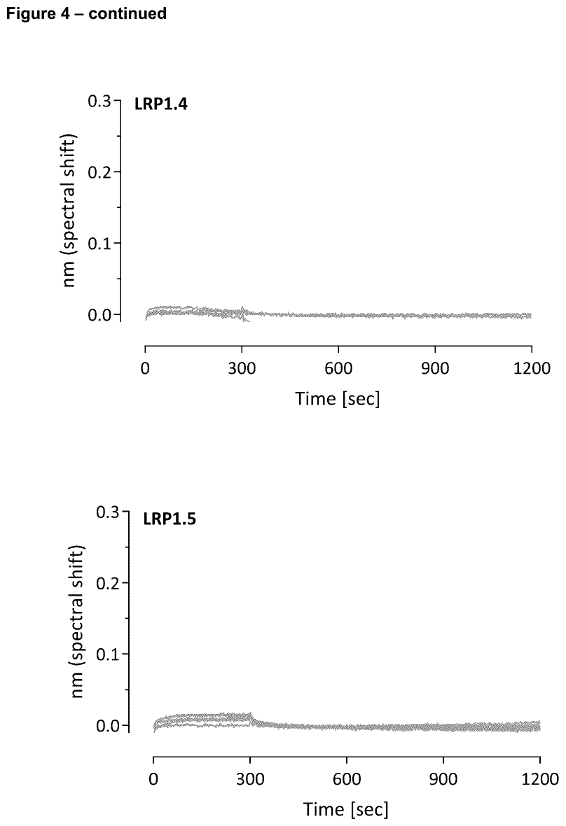

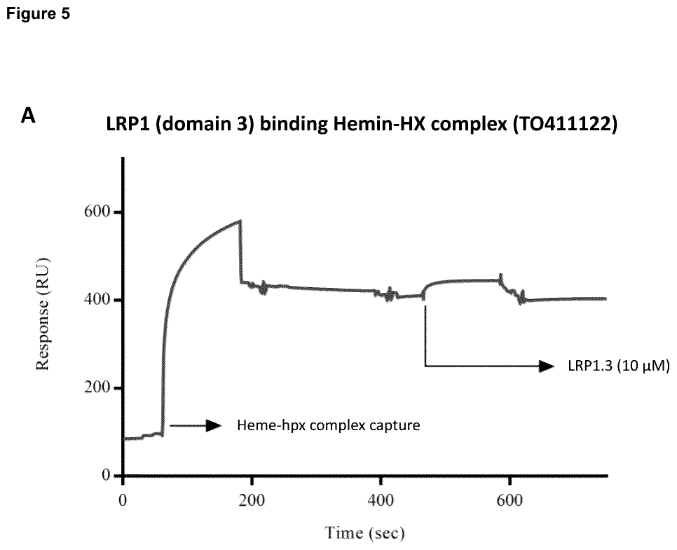

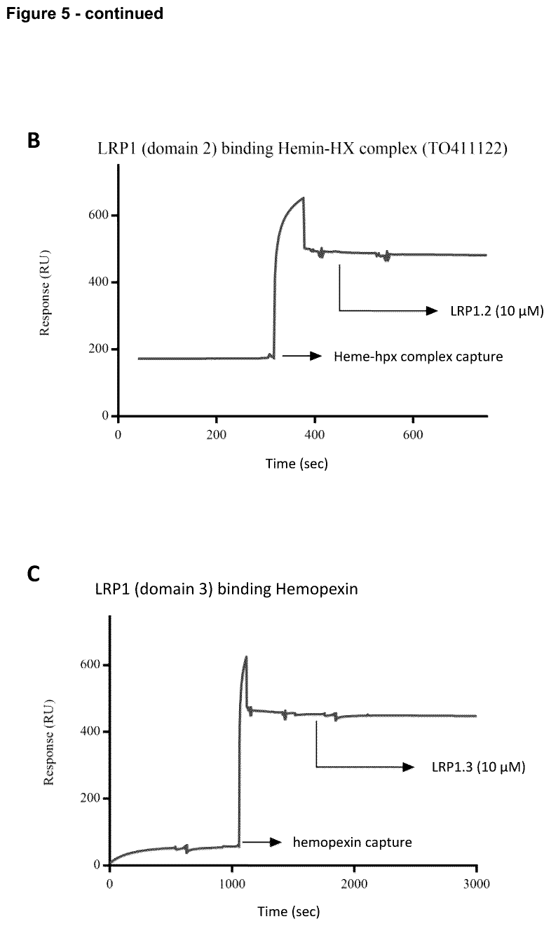

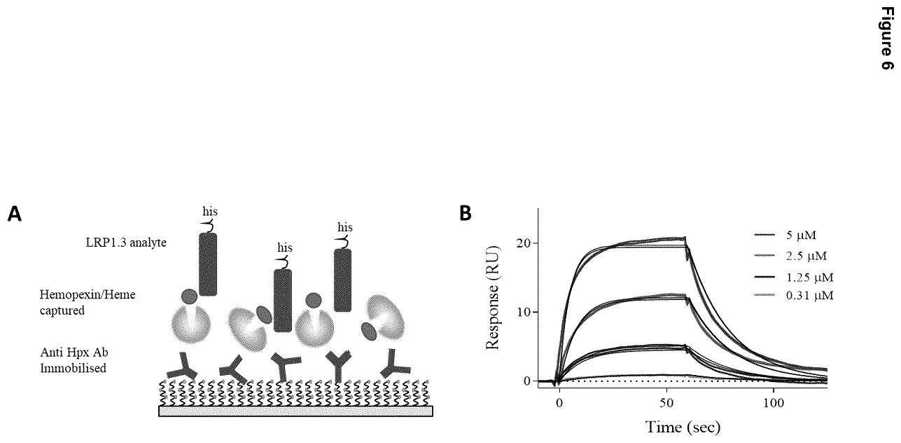

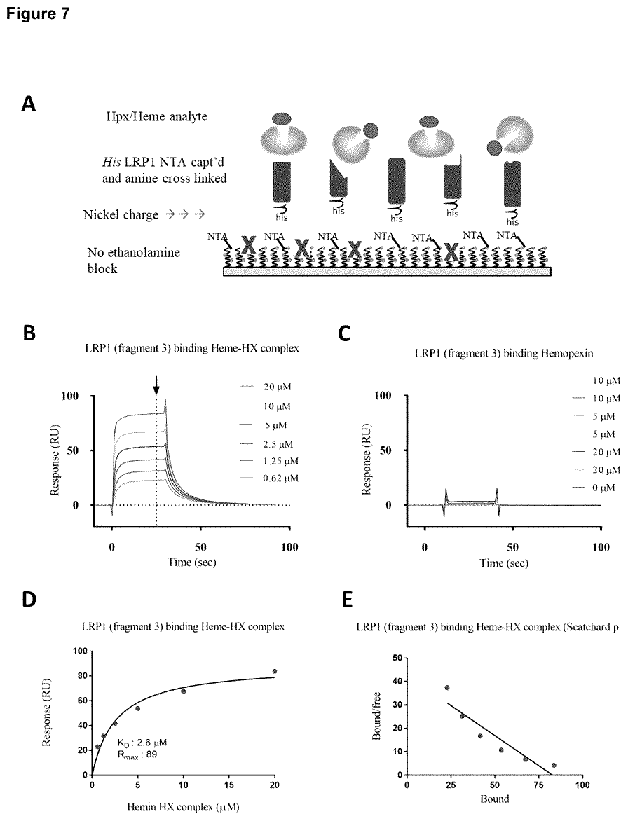

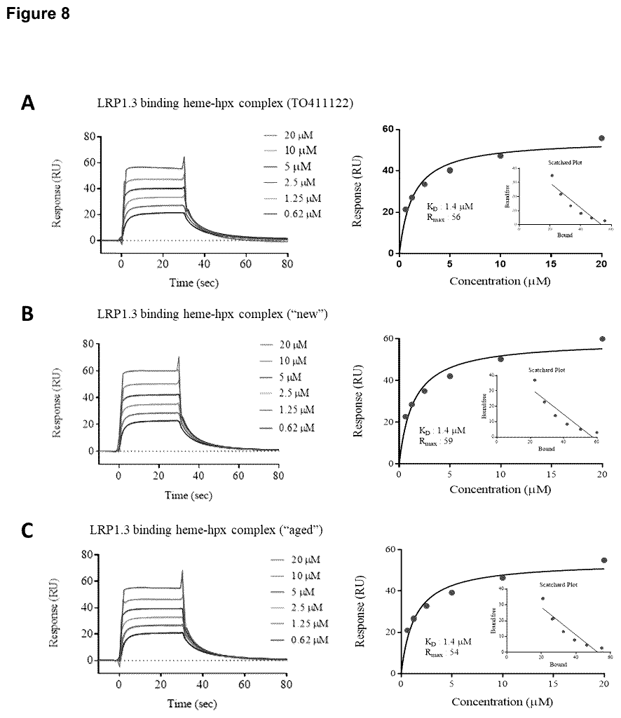

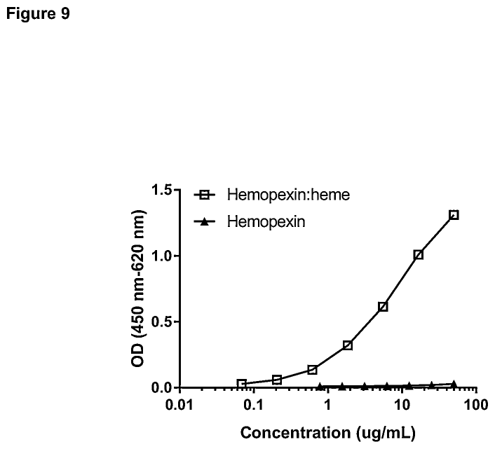

The cDNAs encoding soluble human (hu) LRP1 minireceptors containing each of the 4 ligand binding domains I-IV (Genbank accession number NP_002323.2) and huhLRPAP1 (Genbank Accession number NP_002328.1) were codon-optimized for human expression and synthesized by GeneArt (Invitrogen™, Thermo Fisher Scientific) each with a Kozak consensus sequence (Kozak M., Nucleic Acids Res., 1987; 15(20):8125-48) (GCCACC) immediately upstream of the initiating methionine (+1). The huLRP1 ligand binding domains were based on the boundaries previously described by Obermoeller-McCormick et. al, J Cell Sci, 2001; 114(5):899-908 and illustrated in FIG. 2. Each huLRP1 soluble minireceptor encoded the huLRP1 signal peptide, an N-terminal FLAG tag and a C-terminal 8×His tag fused in-frame and are designated as follows: huLRP1 binding domain I (amino acids 25-113) [huLRP1(1-24)-FLAG-huLRP1(25-113)-8His]; huLRP1 binding domain II (amino acids 806-1183) [huLRP1(1-24)-FLAG-huLRP1(806-1183)-8His]; huLRP1 binding domain III (amino acids 2481-2942) [huLRP1(1-24)-FLAG-huLRP1(2481-2942)-8His]; HuLRP1 binding domain IV (amino acids 3293-3783) [huLRP1(1-24)-FLAG-huLRP1(3293-3783)-8His]. Once each cDNA was completed, it was digested with Nhel and Xhol and ligated into pcDNA3.1 (Invitrogen™, Thermo Fisher Scientific). Large-scale preparations of plasmid DNA were carried out using QIAGEN Plasmid Giga Kits (12191) according to the manufacturer's instructions. The nucleotide sequences of the plasmid constructs were verified by sequencing both strands using BigDye™ Terminator Version 3.1 Ready Reaction Cycle Sequencing (Invitrogen™, Thermo Fisher Scientific.4337455) and an Applied Biosystems 3130xl Genetic Analyzer.

3.3 Transient Transfections for Generation of Recombinant Proteins

Transient transfections of expression plasmids encoding huLRP1 soluble minireceptor binding domains I-IV (90%) together with human LDL Receptor Related Protein Associated Protein 1 (huLRPAP1, RAP, 10%) using ExpiCHO-S™ cells were performed using Expifectamine™ transfection reagent (Invitrogen, Life Technologies) according to the manufacturer's instructions. Cells were transfected at a final concentration of 6×10⁶ viable cells/mL and incubated in a shaking incubator (Infors) for 20 hours at 37° C. in 8% CO₂. After 20 hours, Enhancer™ and a Feed™ was added to the cultures. The culture is then incubated at 32° C., 5% CO₂, 70% humidity for a further 5 days. At day 5 post-transfection a second Feed™ was added to the cultures and they were returned to the incubator at 32° C., 5% CO₂, 70% humidity. The cell culture supernatants were harvested by centrifugation at 2500 rpm and were then passed through a 0.45 μm filter (Nalgene) prior to purification. Expression of recombinant huLRP1 soluble minireceptors in the culture supernatants was confirmed by SDS-PAGE (NuPAGE system, Thermo Fisher Scientific, MA, USA) and also by Western blot analysis using an anti-His antibody (His Tag Antibody [FITC], GenScript, Cat #A01620).

3.4 Purification for HULRP1 Soluble Minireceptors

HuLRP1 soluble minireceptors were purified by tandem Nickel and size exclusion chromatography on an ÄKTA Pure Protein Purification System (GE Healthcare Life Sciences). Samples were applied to a HisPrep™ FF 16/10 Ni+ Sepharose 20 mL column in the presence of 2.5 mM NiCl₂. Column chromatography was generated as per manufacturer's instructions. Post-elution samples were applied directly to a Hi Load 26/600 Superdex 200 pg column (GE Healthcare Bio-Sciences, PA, USA) and eluted into 10 mM Hepes, 150 mM NaCl, pH 7.3. HuLRP1 soluble minireceptor-containing fractions were pooled and concentrated using Amicon Ultra-15 centrifugal filter units 10 kDa (Merck-Millipore, MS, USA) according to the manufacturer's protocol. The concentration of the purified recombinant protein was measured at A280 nm on a Trinean DropSense96 system (Trinean, Gentbrugge, Belgium) and purity of the concentrates verified by SDS-PAGE (NuPAGE system, Thermo Fisher Scientific, MA, USA).

3.5 LRP1 Domains Used for Binding Studies (BLI and SPR)

The following proteins as described in Table 1. were used for binding studies:

TABLE 1
huLRP1 soluble minireceptors used in this study

| Ligand Binding | | DNA | Transfection | Purification | Conc. |
|---|---|---|---|---|---|
| Domain | Protein name | Number | batch | Batch | mg/ml |
| LPR1 domain I | HuLRP1(1-24)-FLAG- | RP3607 | 1LTTC180919.1 | LC Protein | 2.63 |
| (LRP1.1) | HuLRP1(25-113)-8His | | | Samples 4086 | |
| LPR1 domain II | HuLRP1(1-24)-FLAG- | RP3608 | 1LTTC180919.2 | LC Protein | 4.61 |
| (LRP1.2) | HuLRP1(806-1183)-8His | | | Samples 4086 | |
| LPR1 domain III | HuLRP1(1-24)-FLAG- | RP3609 | 1LTTC180919.3 | LC Protein | 2.31 |
| (LRP1.3) | HuLRP1(2481-2942)-8His | | | Samples 4086 | |
| | HuLRP1.3(1-24)-FLAG- | | | LC Protein | 5.73 |
| | HuLRP1(2481-2942)-8His | | | Samples 4204 | |
| | | | | (Pool 1) | |
| LPR1 domain IV | HuLRP1(1-24)-FLAG- | RP3610 | 1LTTC180919.4 | LC Protein | 2.42 |
| (LRP1.4) | HuLRP1(3293-3783)-8His | | | Samples 4086 | |
| LPR1 domain IV | HuLRP1 (3332-3779)-8His, | RP2563 | 1LTTC180919.5 | LC Protein | 1.05 |
| (LRP1.5) | | | | Samples 4086 | |

3.6 Hemopexin Batches Used for Binding Studies (BLI and SPR)

The following hemopexin batches as described in Table 2. were used for binding studies:

TABLE 2
human plasma derived hemopexin used in this study

| Hpx Batch No | Detailed description |
|---|---|
| TO294001 | Heme-hemopexin complex: 119.9 mg/mL |
| TO411122 | Heme-hemopexin complex; 107.8 mg/mL |
| T0342022C | 100 mg/ml (uncomplexed) |
| TO341022BM | Heme-hemopexin complex; 104.75 mg/mL; freshly |
| | purified hemopexin (“new”) |
| TO342022B | Heme-hemopexin complex; 103.30 mg/mL; Hpx stored |
| | for 18 months at 2-8° C. (“aged”) |

3.7 Anti-Penta-HIS (HIS1K) Biosensors

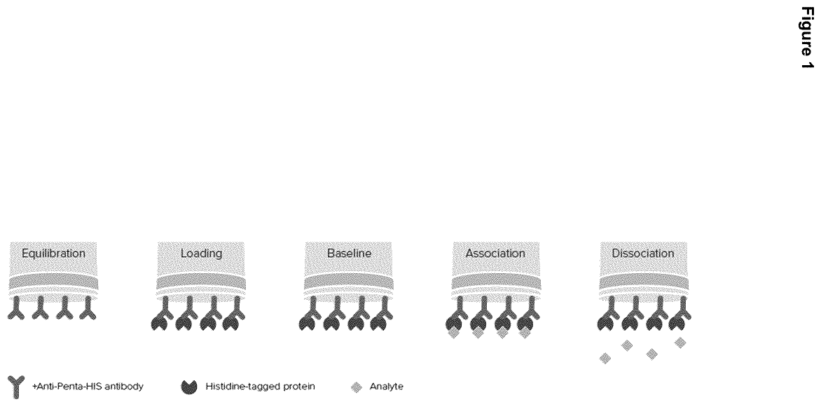

For real-time binding analysis of the heme-hpx complex to immobilized LRP1 soluble minireceptors by BLI, anti-Penta His biosensors were used (FIG. 1), which are pre-immobilized with a highly specific Penta-His antibody. Immobilization of proteins was achieved through direct capture of His-tagged proteins for subsequent kinetic analyses.

3.8 Mini LRP1 Binding Studies Performed on Octet (BLI)

As described above, anti-penta His antibody pre-coated biosensors (Cat N°: 18-5120, ForteBio, TechNote 43: Anti-Penta-HIS (HIS1K) biosensors for label-free analysis of HIS-tagged proteins, 2019) were used. The different LRP1 domains were immobilized at a concentration of 15 μg/mL in 10× kinetic buffer (PBS, 0.1% BSA, 0.02% Tween20). Heme-Hpx complex was diluted in 10× kinetic buffer (PBS, 0.1% BSA, 0.02% Tween20). The association and dissociation kinetics of heme-hemopexin complex, were performed at concentrations of 2.5, 1.25, 0.625 and 0.3125 μM. The settings for each binding step were chosen as shown in the table 3 below. A reference control was included in every experiment (sensor loaded with ligand without analyte). Data was acquired on an OctetRED96 (FortéBio) at 30° C. with the following settings:

TABLE 3
Experimental OctetRED96 settings for kinetic assessment

| Step | Time (s) | Shake speed (rpm) |
|---|---|---|
| Equilibration (buffer) | 100 | 1000 |
| Loading (LRP1; 15 μg/mL) | 600 | 1000 |
| Baseline (buffer) | 60 | 1000 |
| Association (heme-hpx) | 300 | 1000 |
| Dissociation (buffer) | 900 | 1000 |

Data was analyzed by the Data Analysis Software (ForteBio, Version 9.0). Data was processed by performing baseline alignment to the y-axis, inter-step correction, reference sensors subtraction and curve smoothening by Savitzky-Golay Filtering. The processed kinetic dataset was globally fitted using a 1:1 binding model. The fitting accuracy was described by Chi² and R², parameters representing how well the measured results resemble those calculated from the model used to analyze the data.

3.9 Mini LRP1 Binding Studies Performed on Biacore (SPR)

3.9.1 Qualitative Hemopexin Capture Assay

3.9.1.1 LRP1 Domains Binding Heme-Hpx Complex

Rabbit anti-hemopexin polyclonal antibody was directly immobilized onto the carboxymethyl dextran surface of a CM5 sensorchip to approximately ˜18,000 RU using standard NHS/EDC chemistry. Heme-hpx complex made by CSL, Kankakee were captured in flow-cells 2 and 4 respectively at the beginning of each cycle to approximately 350 RU. Reference surfaces (flow-cells 1 and 3) consisted of only anti-hemopexin polyclonal antibody. 10 μM of each fraction (Domains 1, 2 and 4) of LRP1 (see table 1 for details) was injected for 120 seconds over the heme-hpx captured antibody surface.

3.9.1.2 LRP1.3 Binding Heme-Hpx Complex

LRP1.3 (fraction 3, pool-1, sample 4204) was injected in duplicate at concentrations ranging from 0.312 to 20 μM in 2-fold dilution series over the heme-hpx complex captured surface for 30 seconds. The dissociation was monitored for 60 seconds. The antibody surface was pre-conditioned with four injections of 10 mM glycine pH 1.7 for 20 seconds each. Regeneration was done at the end of each cycle using 10 mM glycine pH 1.7 for 20 seconds. Raw data from active surfaces (flow cells 2 and 4) were double referenced by subtraction of signals from reference surfaces (flow cells 1 and 3) and blank injections.

The assay was performed in triplicate at 37° C. in HBS-N supplemented with 5 mM CaCl₂ at pH 7.3. All buffers and solutions were filtered (0.22 μm) prior to use.

3.9.2 NTA Capture Cross Link Assay

Heme-hpx complex binding LRP1.3 was also analyzed using a capture cross linking method using an NTA chip. His tagged LRP1.3 ((fraction 3, pool-1, sample 4204) was tethered to flow cell 4 of an NTA chip using EDC/NHS chemistry. LRP1.3 tethering on the NTA chip was done by forming His tag-nickel-NTA complex on a pre activated surface using EDC/NHS. Flow cell 3, which was treated the same but with no LRP1.3 tethered, was used as a reference cell. Immobilization was done at neutral pH. This method reduces the heterogeneity of the chip surface by orienting the ligand with the His tag. LRP1.3 tethered to around 800 RU with a 1-minute injection at 1 μg/ml.

The capture crosslink protocol method was performed in manual mode according to the following steps:

TABLE 4
Experimental Biacore T200 settings for kinetic assessment

| | | |
|---|---|---|
| Injection step | Repeat | Flow-rate (μL/min) |

| | | | |
|---|---|---|---|
| 350 mM EDTA | 3x 60 | s | 30 |
| 0.5 mM NiCl₂ | 1x 60 | s | 30 |
| EDC/NHS (1:1) | 1x 240 | s | 30 |
| LRP1.3 (1 μg/mL) | 1x 30 | s | 10 |
| 350 mM EDTA | 1x 30 | s | 30 |

The residual amine activity of the surface after tethering was deactivated by running buffer overnight and thereby a chemical deactivation step using ethanolamine was avoided as its was found to affect the ligand activity.

Heme-hpx complex was injected in duplicate at concentrations ranging from 0.62 to 20 μM in 2-fold dilution series over 30 seconds. The dissociation was monitored for 60 seconds. No regeneration step was required between cycles since the complex dissociated fully. Sensogram data from active surface (flow cell 4) were double referenced by subtraction of data from reference surface (flow cells 3) and blank injections. Sensogram data were fit to a 1:1 steady state model.

### 4. Results and Findings

4.1 Expression and Purification of Human LRP1 Soluble Minireceptors

To investigate the hemopexin:heme binding properties of huLRP1 binding domains I, II, III, and IV, we transiently expressed plasmids encoding each of the soluble minireceptors in ExpiCHO-S™ cells (huLRP1 binding domain I (amino acids 25-113), HuLRP1 binding domain II (amino acids 806-1183), huLRP1 binding domain III (amino acids 2481-2942), huLRP1 binding domain IV (amino acids 3293-3783) (FIG. 2). Expression of the huLRP1 soluble minireceptors were performed with and without huLRPAP-1 (RAP) co-expression (FIG. 3A). SDS-PAGE analysis demonstrated that while RAP co-expression is not essential for secretion of huLRP1 minireceptors, there was an approximately two-fold higher expression of the minireceptors with RAP co-expression (FIG. 3A) indicating that co-expression of RAP may facilitate huLRP1 minireceptor secretion (5). SDS-PAGE analysis demonstrated that all the proteins migrated higher than their predicted molecular weights (huLRP1 binding domain I-12.3 kDa, huLRP1 binding domain II-43.6 kDa, huLRP1 binding domain III-53.4 kDa, huLRP1 binding domain IV 57.2 kDa). This is most likely due to glycosylation (FIG. 3A-C).

4.2 Identification of LRp1.3 as the Binding Domain for the Heme-Hpx Complex

To investigate and determine, which of the LRP1 domains bind the heme-hpx complex, each of the domains were tethered on biosensors (HIS1K; anti-penta-His) via the expressed His-tag. As shown in FIG. 4, all four ligand binding domains of LRP1 were tested for their binding to heme-hpx complex. Only LRP1.3 showed definitive binding to the complex. LRP1.3 bound to heme-hpx complex in a dose dependent manner with an affinity of ˜1.5 μM described by a simple 1:1 kinetic model.

TABLE 5
Kinetic rates and fitting parameters (global fit, 1:1 binding model) - LRP1.3

| Domain | Hb [μM] | K_{D} [M] | K_{D} Error | kₒₙ(1/Ms) | k_{off}(1/s) | R² | Chi² |
|---|---|---|---|---|---|---|---|
| LRP1.3 | 2.5-0.312 | 1.46E−6 | 4.94E−8 | 3.79E+04 | 5.55−02 | 0.9907 | 0.0189 |

4.3 Confirmation of IRP1.3 Via a Qualitative Hemopexin Capture SPR Assay

All four domains of LRP1 were tested for their binding to heme-hpx complex by capturing and immobilizing the heme-hpx complex via an immobilized anti-hpx antibody on the chip surface. Assay strategy is outlined in FIG. 6A. As observed with the BLI binding experiment and as shown in FIG. 5A only LRP1.3 showed definitive binding to the complex. No binding towards LRP1.2 is shown. Hemopexin (10 μM) alone showed no significant binding to LRP1 fractions tested (FIG. 5C). Further, LRP1.3 bound to heme-hpx complex was tested in a dose dependent manner with an affinity of ˜2 μM. A concentration dependent secondary component was also observed and data was not well described by a simple 1:1 kinetic model (FIG. 6B). Therefore, an alternative assay setup was used for kinetic parameters estimation.

4.4 Kinetic Parameter Estimation Based on a NTA Capture Cross Link SPR Assay

In a next step and as mentioned above an alternative assay setup was developed as illustrated in FIG. 7A. As shown in FIG. 7B heme-hpx complex bound LRP1.3 in a dose dependent way and a reproducible K_{D} could be calculated by steady state affinity of ˜2 μM as summarized in Table 6.

TABLE 6
Calculated steady state binding affinity of heme-hpx complex binding
LRP1.3. K_{D} indicated as Mean ± SEM (N = 3). Values were
calculated from sensorgram data fit to a 1:1 steady state binding model.

| Experiment/Run | K_{D} (μM) | Rₘₐₓ (RU) |
|---|---|---|
| 1 | 2.62 | 89.3 |
| 2 | 2.61 | 89.0 |
| 3 | 2.63 | 88.8 |
| Mean | 2.62 | 89.03 |
| SEM | 0.006 | |

As determined, the affinity of heme-hpx complex binding LRP1.3 was reported at ˜2.6 μM. Additional complexes were tested in this assay setup and all were showing similar steady state affinity of approximately 1.4 μM (Table 7 and FIG. 8).

TABLE 7
Calculated steady state binding affinity of heme-hpx complex binding
LRP1.3.Assay was conducted N = 1. Values were calculated
from sensogram data fit to a 1:1 steady state binding model.

| Heme-hpx complex | K_{D} (μM) | Rₘₐₓ (RU) |
|---|---|---|
| T0342022C | 1.34 | 59 |
| TO341022BM (“New”) | 1.41 | 56 |
| TO342022B (“Aged”) | 1.35 | 54 |

### 5. Discussion and Conclusion

The ability to express the different LRP1 immunoreceptors (domains) facilitated the identification of the heme-hemopexin binding domain in vitro. In summary we could demonstrate with different methodologies (BLI and SPR) and different capture strategies that CD91/LRP1 domain III is responsible to bind heme-hemopexin complexes. The binding is highly specific once hemopexin is complexed with a heme molecule, since uncomplexed hemopexin (apoHpx) does not bind, which was again confirmed on both platforms used within this study. It was further found that none of the CD91/LRP1 domains I, II, and IV bind the heme-hemopexin complexes.

Further, we could demonstrate similar binding kinetics independent of the platform and hemopexin batch used as summarized in Table 8 for LRP1.3

TABLE 8
Summary of all calculated kinetic parameters of heme-hpx complex
binding to LRP1.3. Values were calculated based on sensogram data
fitted to a 1:1 binding model. Calculation method is indicated.

| | | K_{D} | Rₘₐₓ | Calculation |
|---|---|---|---|---|
| Platform | Heme-hpx complex | (μM) | (RU) | method |
| BLI | TO294001 | 1.46 | n/a | Kinetic fit |
| SPR | TO411122 | ~2 | n/a | Kinetic fit |
| | | | | (not optimal) |
| SPR | TO411122 | 2.62 | 89 | Steady state |
| SPR | T0342022C | 1.34 | 59 | Steady state |
| SPR | TO341022BM (“New”) | 1.41 | 56 | Steady state |
| SPR | TO342022B (“Aged”) | 1.35 | 54 | Steady state |

### 6. Heme-Hemopexin Complex Preparation

Preparation of Heme-Hemopexin (Heme-Hx) Complex
1. Dilute hemopexin (100 mg/mL) with PBS pH 7.4, to approximately 28-31 mg/mL (˜460-509 μM) (final volume=batch size dependent). Note: hemopexin concentrate does not require a dilution since it is already in PBS pH 7.4 at ˜30 mg/mL.
2. 0.2 μM filter the PBS-diluted hemopexin into a sterile or heat-shocked bottle equipped with a stir bar while in a laminar flow hood.
3. Remove 10 mL and place into a sterile vial. Set aside for use as a control without hemin.
4. Determine the μM concentration of the PBS-diluted hemopexin using a molecular weight of 60,888.
5. Prepare Hemin (Frontier Scientific, Cat no: H651-9, m.w.=651.94) in 100% DMSO (Sigma-Aldrich, Cat no: D2438-10 mL, Sterile-Filtered, Bioperformance Certified, meets EP, USP testing specifications) at a concentration (usually ˜15-17 mg/mL) at which a 1/50 dilution will result in the same μM concentration as determined for the hemopexin. Prepare just before use. Final volume depends on hemopexin batch size.
6. Add the volume of hemin needed to dilute the hemin 1/50 in the hemopexin. This should result in equal μM of hemopexin and heme (e.g. 500 μM+500 μM) for a 100% saturation target. This should be done while still under the laminar flow hood.
7. Cap and mix immediately by inversion and swirling.
8. Dilute DMSO 1/50 in the vial containing the 10 mL of hemopexin from step c). This is used as a control and as a blank for determining the percent saturation.
9. Incubate and gently stir both the complex and the control overnight at 37° C. in an incubator equipped with a stir plate to allow heme-hpx complex formation to proceed.
10. Pull a 1 mL sample for saturation analysis.
11. Prepare two dilutions of the complex and the control in 10% DMSO in PBS to approximately 0.45 mg/mL and 0.25 mg/mL based on the initial hemopexin concentration of 28-31 mg/mL. This should result in 414 nm absorbance values of ˜0.83 and ˜0.45
12. Determine the percent saturation at 414 nm using the control as a blank.
13. Calculate the μM of heme-hemopexin complex at each concentration using the extinction coefficient of 123.1.
14. Divide the μM of complex by the μM of hemopexin in the reaction and multiply by 100 to get the % saturation. Average the two readings for the final saturation percentage.

Ultrafiltration and Diafiltration of Heme-Hemopexin Complex
1. 0.2 μm filter the heme-hx complex.
2. Ultrafilter to target concentration/volume using a BioMax 10 kd mwco Millipore Pellicon cassette.
3. Diafilter against 10× volume of hemopexin FIN buffer (0.9 mM Citrate, 14.1 mM S. Phosphate, 150 mM NaCl, pH 7.2).
4. Ultrafilter back to final target volume
5. Pull retains for analysis.
6. Dilute to approximately 0.45 mg/mL and 0.25 mg/mL to determine the μM and mg/mL of heme-hx complex which can then be used to back calculate the hemopexin mg/mL using the saturation percentage.
7. Aliquot and label tubes.
8. Freeze at ≤−65 C.

### Example 2. Hemopexin:Heme Complex and Hemopexin-Accessible Heme Assay Design

### 1. Introduction

The purpose of the following experiments is to determine whether 1-24-FLAG-HuLRP1-domain III (CD91-domain III) can be used as a coating reagent to measure hemopexin:heme complexes in biological samples. For this purpose, this construct, which binds specifically to hemopexin:heme complexes is applied as a coating reagent to a Maxisorp ELISA plate. Following incubation, the LRP1-coated plate is washed and then blocked with 1% casein in PBS to reduce non-specific binding. After blocking, the plate is washed, and each sample is added to the plate in two formats:
1. Serum samples diluted 50 fold and assayed to measure hemopexin:heme complex.
2. Un-spiked (quantifying endogenous complex concentrations) and spiked with Hemopexin that will form complexes with any free heme in the sample (in order to measure hemopexin-accessible heme).

Sample type 2 may be omitted if quantifying hemopexin:heme complex only.

The samples referred to within this Example 2 are serum samples which have been obtained from either heathy individuals or sickle cell disease patients.

Following sample incubation, the plates are washed again and incubated with the primary detection antibody anti-hemopexin rabbit polyclonal antibody (Abcam, Ab48135). Plates are washed before the addition of secondary detection antibody goat anti-rabbit horse radish peroxidase (HRP) conjugated antibody. Following another incubation, tetramethlybenzidine (TMB) is applied to each well, which reduces the hydrogen peroxidase in HRP causing it to change colour to blue. The reaction is stopped with the addition of sulphuric acid, causing the reduced hydrogen peroxidase to change colour to yellow, the intensity of which is proportional to the concentration of hemopexin:heme complexes present in the sample.

Plates are read at 450 nm (620 nm reference) using a plate reader. The concentration of hemopexin:heme complex from each sample is interpolated from the standard curve. The relative, quasi-quantitative concentration of hemopexin-accessible heme can be determined by subtracting the concentration of hemopexin:heme complex present in the un-spiked samples (A) from hemopexin spiked samples (B). The resulting delta hemopexin:heme complex concentration (B-A) is divided by the molecular weight of hemopexin. Heme binds to hemopexin at a 1:1 ratio, therefore the resulting concentration after dividing the delta value by the molecular weight of hemopexin is the relative quasi-molarity of the hemopexin accessible heme within a sample (C).

Relative accessible heme concentration (C)=([spiked (B)−unspiked (A)]/63000)*1000 (μM)

### 2. Equipment and Software

Calibrated variable-volume single channel pipettes capable of delivering 1 μL to 1 mL
Calibrated variable-volume multi-channel pipettes capable of delivering 1 μL to 300 μL
Plate reader capable of reading at A 450 nm (Tecan M200 or equivalent)
Magellan Software V 7.2
Microsoft Excel
Plate shaker (Micromix 5 or equivalent)
Vortex Mixer
Timer
96 Well Plate Washer (must be capable of performing 5 washes, dispensing 300 μL per well)

### 3. Materials

96F Maxisorp NUNC ELISA plates (Thermo Fisher Cat No. 439454)

### 4. Reagents

1-24-FLAG-HuLRP1-domain III (CD91-domain III)
Hemopexin:Heme Complex
Hemopexin
Abcam Anti-Hemopexin rabbit polyclonal antibody primary detection (1.056 mg/mL) Cat No. Ab48135.
Goat anti-rabbit horse radish peroxidase (HRP) antibody secondary detection (Seracare Cat No. 5220-0336)
Blocking buffer/assay diluent 1% Casein in PBS (Thermo Fisher Cat No. 37528)
Dulbecco's Phosphate-buffered saline (DPBS) (Sigma, Cat. #D8537)
Wash Buffer: 1×PBS/Tween
Substrate: TMB A & B (KPL Cat No. 5120-0049 and 5120-0038)
Stop solution: Sulphuric acid (0.5M) (Thermo fisher Cat No. 124240010)

### 5. Operation/Procedure

5.1 Preparation of Reagents
1-24-FLAG-HuLRP1-domain III (CD91-domain III)
   Required at 5 μg/mL; dilute in D-PBS
   11 mL is required per plate
Primary detection antibody: Anti-hemopexin rabbit polyclonal
   Required at 1 μg/mL; dilute in blocking buffer/assay diluent (1% Casein in PBS)
   11 mL is required per plate
Secondary detection antibody: goat anti-rabbit HRP
   Stock reagent is required to be diluted 1/2000, dilute in blocking buffer/assay diluent (1% casein in PBS)
   i.e. 5.5 μL of HRP into 10994.5 μL of blocking buffer/assay diluent
Substrate: TMB A & B
   Combine TMB solution A and B together in equal volumes immediately prior to use.
   12 mL is required per plate
   i.e. 6 mL TMB A+6 mL TMB B

5.2 Preparation of Standards

The standard curve is prepared from hemopexin:heme complex. The following pre-dilution (PD) steps will be performed in blocking buffer/assay diluent (1% casein in PBS).

TABLE 9
Standard Pre-Dilutions

| | | Hemopexin:Heme | |
|---|---|---|---|
| Standard | | Complex concentration | |
| Pre-Dilution | Stock Used | (μg/mL) | Dilution Factor |
| PD 1 | Lot T0411122 | 1000 | 104.75 |
| | (104.75 mg/mL) | | |

The standard curve is then prepared by serially diluting 2-fold PD 1 in blocking buffer/Assay diluent (1× Casein in PBS) on the day of assay as shown in Table 2.

TABLE 10
Standard curve

| | | | | | Assay | |
|---|---|---|---|---|---|---|
| | | | | Stock | Diluent | Total |
| | Concentration | Stock | | Volume | Volume | Volume |
| Standard | (μg/mL) | to Use | DF | (μL) | (μL) | (μL) |
| 1 | 100 | PD 1 | 10 | 50 | 450 | 500 |
| 2 | 50 | 100 | 2 | 250 | 250 | 500 |
| 3 | 25 | 50 | 2 | 250 | 250 | 500 |
| 4 | 12.5 | 25 | 2 | 250 | 250 | 500 |
| 5 | 6.25 | 12.5 | 2 | 250 | 250 | 500 |
| 6 | 3.125 | 6.25 | 2 | 250 | 250 | 500 |
| 7 | 1.563 | 3.125 | 2 | 250 | 250 | 500 |
| 8 | 0.781 | 1.563 | 2 | 250 | 250 | 500 |
| 9 | 0.391 | 0.781 | 2 | 250 | 250 | 500 |
| 10 | 0.195 | 0.391 | 2 | 250 | 250 | 500 |

Note
This is sufficient volume for 1 plate and may be scaled up as required.
DF; dilution factor.
Standard 1 and 10 are anchor points.

5.3 Preparation of Controls

The positive control (PC) is hemopexin:heme complex. The PCs will be prepared as per Table 3 in 100% pooled human serum. The negative control (NC) will be the same 100% pooled human serum used in the preparation of the PCs.

TABLE 11
Control preparation

| | | | | | 100% | | |
|---|---|---|---|---|---|---|---|
| | | | | | pooled | | On- |
| | | | | Stock | human | Total | plate |
| | Stock conc. | Stock | | Volume | serum | volume | conc. |
| Control | (μg/mL) | to use | DF | (μL) | (μL) | (μL) | (μg/mL) |
| PC | 500 | 104.75 mg/mL | 209.5 | 11.93 | 1238.1 | 2500 | 10 |
| NC | 0 | N/A | N/A | N/A | 1500 | 1500 | 0 |

Apply a 50 fold dilution to the PC and NC prior to plating i.e. 5 μL of control+245 μL blocking buffer/assay buffer.

5.4 Test Sample Preparation
Test samples should be thawed at room temperature before assaying. Samples are stable at room temperature (RT) for up to 24 hours as determined during verification. Test samples may be assayed only as Hemopexin:heme complex (sample type 1), or as both Hemopexin:heme complex (1) and Hemopexin:accessible heme (sample type 2).
Hemopexin:heme complex (sample type 1) readout:
   Dilute serum samples 50 fold in blocking buffer/assay diluent.
      i.e. 5 μL of control into 245 μL blocking buffer/assay diluent.
Hemopexin-accessible heme (sample type 2) readout:
   Spike each test sample with 500 μg/mL of Hemopexin as per Table 4. Incubate at room temperature for 30 minutes.
   The un-spiked sample must be diluted as per the final Hemopexin spike with DPBS.
   i.e. 2.5 μL of DPBS into 47.5 μL of serum. Note: this must be done to adjust the un-spiked samples endogenous complex concentrations by the same dilution factor as the spiked samples.
   Apply a 50 fold dilution to the spiked sample i.e. 5 μL of control into 245 μL blocking buffer/assay diluent.

TABLE 12
Hemopexin-accessible heme hemopexin sample spiked and un-spiked preparation

| | | D-PBS | Test sample | Total |
|---|---|---|---|---|
| | | volume | volume | volume |
| | DF | (μL) | (μL) | (μL) |
| Sample un-spiked adjustment dilution | 20 | 2.5 | 47.5 | 50 |

| | Hemopexin | | | Stock | Test sample | Total |
|---|---|---|---|---|---|---|
| Sample | Concentration | Stock | | Volume | volume | volume |
| Spike | (μg/mL) | to use | DF | (μL) | (μL) | (μL) |
| Intermediate | 10,000 | 98.35 mg/mL | 9.835 | 5.1 | 44.9 | 50 |
| Dilution | | | | | | |
| Hpx Spike | 500 | Intermediate | 20 | 2.5 | 47.5 | 50 |
| | | Dilution | | | | |

5.5 Assay Procedure
1. Coat Maxisorp NUNC 96 well plate with 100 μL per well with the capture coating solution. Cover plate with a plate sealer and incubate, shaking at RT for 1 hr (±5 minutes).
2. Wash plate 5× with at least 200 μL per well of 1×PBS/Tween wash buffer (Plate washer or manually).
3. Block the plate with 200 μL per well of Blocking buffer/Assay Diluent (1% casein in PBS). Cover plate with a plate sealer and incubate, shaking at RT for 1 hr (±5 minutes).
4. Wash plate 5× with 1×PBS/Tween wash buffer (Plate washer or manual).
5. Add 100 μL of standard/controls/samples to the appropriate wells. Cover plate with a plate sealer and incubate, shaking at RT for 1 hr (±5 minutes).
6. Wash plate 5× with 1×PBS/Tween wash buffer (Plate washer or manual).
7. Add 100 μL per well of anti-hemopexin rabbit polyclonal antibody primary detection solution. Cover plate with a plate sealer and incubate, shaking at RT for 1 hr (±5 minutes).
8. Wash plate 5× with 1×PBS/Tween wash buffer (Plate washer or manual).
9. Add 100 μL per well of the goat anti-rabbit HRP secondary detection. Cover plate with a plate sealer and incubate, protected from light, shaking at RT for 1 hr (±5 minutes).
10. Wash plate 5× with 1×PBS/Tween wash buffer (Plate washer or manual).
11. Add 100 μL per well of substrate and incubate, shaking at RT for 3.5 minutes or until optimal color change. Add 50 μL per well of stop solution (Sulphuric acid 0.5M) in the same pipetting orientation as TMB was added.
12. Read plate on a plate reader at 450 nm (620 nm reference) within 10 minutes on adding stop solution.

### 6. Validation Data for Hemopexin:Heme Assay and Hemopexin-Accessible Heme Assays

6.1 System Suitability
1. Ten separate assays were conducted with a 10-point standard curve plated in duplicates. Standard curves were fitted using Magellan software with a 5-parameter logistic curve fit with an R value generated, CV and RE calculated in Excel.
2. As shown below, 8 of the 10 validation plates generated an acceptable % CV and % RE within 25%. Therefore, the assay standard curve was considered suitable for a fit-for-purpose assay.

TABLE 13
Co-efficient of variation (CV) of interpolated standards (10 assay runs).

| | Complex | | | | | | | | | | |
|---|---|---|---|---|---|---|---|---|---|---|---|
| | Expected | | | | | | | | | | |
| | conc. | | Validation run, % CV | | | | | | | | |
| Standard | (μg/mL) | 1 | 2 | 3 | 4 | 5 | 6 | 7 | 8 | 9 | 10 |
| 1 | 100 | N/A | N/A | 15 | 6 | 7 | N/A | 11 | 26 | N/A | N/A |
| 2 | 50 | N/A | 7 | 24 | 29 | 14 | 10 | 2 | N/A | 56 | 61 |
| 3 | 25 | 2 | 12 | 11 | 13 | 1 | 3 | 24 | 3 | N/A | 5 |
| 4 | 12.5 | N/A | 10 | 6 | 14 | 7 | 5 | 10 | 9 | 12 | 1 |
| 5 | 6.25 | 9 | 4 | 5 | 4 | 3 | 7 | 5 | 16 | 9 | 3 |
| 6 | 3.125 | 9 | 3 | 1 | 0 | 2 | 5 | 1 | 6 | 2 | 2 |
| 7 | 1.563 | 32 | 4 | 1 | 5 | 5 | 0 | 2 | 0 | 1 | 0 |
| 8 | 0.7813 | 0 | 1 | 1 | 8 | 4 | 6 | 2 | 0 | 1 | 9 |
| 9 | 0.3906 | 7 | 7 | 6 | 1 | 5 | 7 | 1 | 1 | 2 | 15 |
| 10 | 0.1953 | Min | 4 | 4 | Min | Min | Min | Min | Min | Min | Min |

| | | | | | | | | | | |
|---|---|---|---|---|---|---|---|---|---|---|
| Blank | Min | Min | Min | Min | Min | Min | Min | Min | Min | Min |
| R Value | 0.998 | 0.999 | 0.999 | 0.999 | 0.999 | 0.999 | 0.999 | 0.999 | — | 0.999 |

Grey cells - anchor points; no acceptance criteria apply. Italics - failed results (note Plate 1 and 9 failed); N/A- <2 data points therefore CV could not be determined; Min - OD values below the software's capacity to determine. — R value not determined.

TABLE 14
Relative error (% RE) of experimentally determined concentration compared
to the expected concentration of interpolated standards (10 assay runs).

| | Complex | | | | | | | | | | |
|---|---|---|---|---|---|---|---|---|---|---|---|
| | Expected | | | | | | | | | | |
| | conc. | Validation run, % RE | | | | | | | | | |
| Standard | (μg/mL) | 1 | 2 | 3 | 4 | 5 | 6 | 7 | 8 | 9 | 10 |
| 1 | 100 | −58 | −15 | −22 | −14 | −8 | −47 | −20 | −33 | −60 | −17 |
| 2 | 50 | 30 | −21 | 10 | 19 | 8 | 34 | 7 | −1 | −12 | 0 |
| 3 | 25 | −8 | 3 | 11 | −1 | 0 | 0 | 9 | −4 | 13 | 9 |
| 4 | 12.5 | 7 | 3 | −1 | −1 | −1 | 0 | 1 | 3 | 23 | 11 |
| 5 | 6.25 | −8 | 3 | −4 | −1 | 0 | −2 | −2 | 0 | −9 | −3 |
| 6 | 3.125 | −2 | −2 | 0 | 2 | 0 | 0 | −1 | −2 | 0 | −3 |
| 7 | 1.563 | 23 | −8 | 5 | −1 | 3 | 5 | 2 | 4 | 2 | 2 |
| 8 | 0.7813 | −3 | 2 | 8 | 1 | 2 | 7 | 3 | 0 | 3 | 7 |
| 9 | 0.3906 | −10 | 5 | 11 | 5 | −4 | −7 | −1 | 0 | 3 | 11 |
| 10 | 0.1953 | Min | 16 | 5 | Min | Min | Min | Min | 3 | Min | Min |

| | | | | | | | | | | |
|---|---|---|---|---|---|---|---|---|---|---|
| Blank | Min | Min | Min | Min | Min | Min | Min | Min | Min | Min |

Grey cells - anchor points; no acceptance criteria apply. Italics - failed results. N/A - <2 data points therefore CV could not be determined; Min - OD values below the software's capacity to determine.

### 7. Specificity

Assay specificity was tested to assess cross-reactivity between free hemopexin (apo-hemopexin) and hemopexin:heme complex. A 7-point standard curve was prepared using either Hemopexin:heme complex or free hemopexin. As shown in FIG. 9, the assay is specific for hemopexin bound to heme and does not bind free hemopexin.

### 8. Intermediate Precision/Repeatability

The 5 positive controls generated from hemopexin:heme complex (UHPC, HPC, MPC, LPC, ULPC) and the negative control (NC) were assessed for repeatability (intra assay; 3 sets of triplicates in one assay run), inter-assay (3 independent assay runs by one operator) and inter-operator precision (two operators performing two assay runs each).

As shown in Tables 6, 7 and 8, negative controls had CV<25% for all parameters. As shown in Tables 6, 7 and 8, the HPC, MPC, LPC and ULPC demonstrated acceptable precision as % CV were <25%.

TABLE 15
Repeatability (intra-assay precision). Three individual sets of controls
assayed on one plate on a single day. Measured concentrations are averaged
from duplicate wells, following background (NC) subtraction.

| | | | | | Intra-assay precision | | |
|---|---|---|---|---|---|---|---|
| | | | | | Mean | | |
| | Expected on | Mean measured conc. | | | measured | | |
| PC and | plate conc. | (NC subtracted, μg/mL) | | | conc. | | |
| NC | (μg/mL) | Set 1 | Set 2 | Set 3 | (μg/mL) | % CV | % RE |
| UHPC | 30 | 33.0 | 52.2 | 29.0 | 38.1 | 33 | 27 |
| HPC | 20 | 26.7 | 25.4 | 19.6 | 23.9 | 16 | 19 |
| MPC | 10 | 10.0 | 10.1 | 8.6 | 9.5 | 9 | −5 |
| LPC | 4 | 4.0 | 3.9 | 3.8 | 3.9 | 2 | −2 |
| ULPC | 1 | 1.1 | 1.0 | 1.0 | 1.0 | 6 | 1 |
| NC | | 1.1 | 1.3 | 1.1 | 1.2 | 10 | |

Italics - Failed results. Grey cells, N/A

TABLE 16
Inter-assay precision - 3 independent assay runs of controls performed
by one operator (KY). Measured concentrations were averaged from
duplicate wells, following background (NC) subtraction.

| | | | | | Inter-assay | | |
|---|---|---|---|---|---|---|---|
| | | | | | Mean | | |
| | Expected on | Mean measured conc. | | | measured | | |
| PC and | plate conc. | (NC subtracted, μg/mL) | | | conc. | | |
| NC | (μg/mL) | Plate 1 | Plate 2 | Plate 3 | (μg/mL) | % CV | % RE |
| UHPC | 30 | 67.3 | 37.9 | 44.5 | 49.9 | 31 | 66 |
| HPC | 20 | 21.0 | 21.5 | 20.8 | 21.1 | 2 | 6 |
| MPC | 10 | 10.6 | 11.0 | 9.3 | 10.3 | 9 | 3 |
| LPC | 4 | 4.0 | 3.9 | 3.6 | 3.8 | 5 | −4 |
| ULPC | 1 | 1.1 | 1.0 | 1.0 | 1.0 | 7 | 0 |
| NC | | 1.4 | 1.0 | 1.3 | 1.2 | 16 | |

Italics - Failed results. Grey cells, N/A

TABLE 17
Inter-operator precision - two operators (KY, HM) performed 2 assay runs. Measured concentrations
for controls was averaged from duplicate wells, following background (NC) subtraction.

| | | | | | | Intra-assay | | |
|---|---|---|---|---|---|---|---|---|
| | | Mean measured conc. | | | | Mean | | |
| | Expected on | (NC subtracted, μg/mL) | | | | measured | | |
| PC and | plate conc. | KY Set 1 | KY Set 2 | HM Set 1 | HM Set 2 | conc. | | |
| NC | (μg/mL) | (Plate 2) | (Plate 4) | (Plate 3) | (Plate 5) | (μg/mL) | % CV | % RE |
| UHPC | 30 | 37.9 | 44.5 | 33.0 | 58.4 | 43.5 | 25 | 45 |
| HPC | 20 | 21.5 | 20.8 | 26.7 | 28.6 | 24.4 | 16 | 22 |
| MPC | 10 | 11.0 | 9.3 | 10.0 | 10.9 | 10.3 | 8 | 3 |
| LPC | 4 | 3.9 | 3.6 | 4.0 | 4.1 | 3.9 | 6 | −2 |
| ULPC | 1 | 1.0 | 1.0 | 1.1 | 1.1 | 1.0 | 6 | 1 |
| NC | | 1.0 | 1.3 | 1.1 | 1.7 | 1 | 22 | |

Italics - Failed results. Grey cells, N/A

### 9. Accuracy

Accuracy was assessed using the same data generated for precision (Section 7.3). Accuracy of the controls were calculated after each sets NC (background) subtraction. As shown in Tables 6, 7 and 8, the HPC, MPC, LPC and ULPC have demonstrated acceptable accuracy (RE±≤25% for HPC, MPC, LPC and ±≤30% for and ULPC). The UHPC failed its accuracy criteria (RE±30%) and as discussed further in range (Section 7.5) reduces the assays upper limit of quantification to the HPC concentration (20 μg/mL on plate).

### 10. Range and Quantitation Limit

Pooled healthy serum controls spiked with known quantities of hempexin:heme spiked controls in conjunction with the standard curve were used to evaluate assay range by assessing the highest and lowest concentrations that adhere to CV≤25% and RE±≤30%. The effective range of quantification for the assay is based on the precision and accuracy of the spiked controls. The upper limit of quantification of the assay has been determined to be the 1000 μg/mL (20 μg/ml on plate) and the lower limit of quantification to be 50 μg/mL (1 μg/mL on plate). To determine the lowest limit of detection for the assay, hemopexin:heme complex was spiked into pooled human serum at 500 μg/mL at a 50 fold dilution of the sample. Following the 50 fold dilution, the spiked sample was serial diluted before assaying alongside an un-spiked similarly-diluted serum sample (see table below).

TABLE 18
Range - Hemopexin:heme complex was spiked into serum at 500
μg/mL. Both spiked and unspiked serum was diluted 50 fold
followed by serial dilutions thereafter and the on-plate calculated
concentration of each spiked sample dilution was generated
by interpolation from the standard curve, with the un-spiked
sample diluted at MRD used to apply background subtraction.

| | On Plate | | Background | | |
|---|---|---|---|---|---|
| | Complex | Calc | Subtracted | | |
| | Spike Conc | Conc | Calc Conc | | |
| Dilution | (μg/mL) | (μg/mL) | (μg/mL) | % RE | RE ≤30% |
| Dil 1 | 10 | 16.65 | 16.03 | 60 | No |
| Dil 2 | 6 | 6.98 | 6.36 | 6 | Yes |
| Dil 3 | 3 | 3.68 | 3.06 | 2 | Yes |
| Dil 4 | 2 | 2.67 | 2.05 | 3 | Yes |
| Dil 5 | 1 | 1.63 | 1.01 | 1 | Yes |
| Dil 6 | 0.8 | 1.41 | 0.79 | −1 | Yes |
| Dil 7 | 0.6 | 1.25 | 0.62 | 4 | Yes |
| Dil 8 | 0.4 | 1.02 | 0.40 | 0 | Yes |
| Dil 9 | 0.2 | 0.88 | 0.26 | 30 | Yes |
| Dil 10 | 0.15 | 0.77 | 0.15 | 0 | Yes |
| Dil 11 | 0.1 | 0.76 | 0.14 | 37 | No |
| | 0 | 0.62 | | | |

As demonstrated above, acceptable RE (≤30%) was achieved at 0.15 μg/mL on-plate concentration. Therefore, the assays range of quantification is determined as 50-1000 μg/mL in 100% human serum (1-20 μg/mL on plate) based upon the performance of the controls. However, based on the range data; acceptable detection was achieved down to 0.15 μg/mL on plate following background subtraction. Therefore, the assays limit of the detection for hemopexin:heme complex is 0.15 μg/mL on plate concentration following background subtraction. Concentrations between 0.15 and 1 μg/mL can be reported with a degree of uncertainty applied.

### 11. Selectivity of the Hpx-Accessible Heme Assay

For the hemopexin-accessible heme selectivity, individuals were first assessed by spiking with hemopexin to generate additional complexes with free heme within each sample. There relative hemopexin-accessible heme concentration was calculated as:

Relative hemopexin-accessible heme concentration (μM) (C)=([spiked (B)−unspiked (A)]/63000)*1000

Results indicated that while individuals have a basal level of hemopexin:heme complexes in serum, free heme is relatively rare in tested samples.

TABLE 19
Selectivity for hemopexin-accessible heme assay. Healthy, SCD serum/plasma
and haemolysed serum individual donor samples were assayed (A) un-spiked,
or (B) spiked (with 500 μg/mL of hemopexin). Un-spiked and spiked measured
hemopexin:heme complex concentrations were back-calculated by from the 50
fold dilution. (A) un-spiked complex concentrations (endogenous/background)
were subtracted from (B) spiked complex concentrations (background subtracted)
to find (C) delta complex concentrations. Accessible heme was calculated
from (C). Precision (% CV) is calculated for duplicate wells.

| | Mean back calculated Hemopexin:heme Complex | | | | | Accessible |
|---|---|---|---|---|---|---|
| | (A) Un-spiked | | (B) Hpx spiked | | | Heme |
| | mean back | | mean back | | Delt conc | (C) Relative |
| | calc conc | | calc conc | | (B − A) | Molarity |
| Donor | (μg/mL) | % CV | (μg/mL) | % CV | (μg/mL) | (μM) |
| 1 SCD serum | 18.77 | 8 | 21.80 | 11 | 3.03 | 0.05 |
| 2 SCD serum | 15.51 | 10 | 18.22 | 5 | 2.71 | 0.04 |
| 3 SCD serum | 41.63 | 0 | 44.42 | 2 | 2.79 | 0.04 |
| 1 SCD plasma | 16.00 | 5 | 18.87 | 3 | 2.88 | 0.05 |
| 2 SCD plasma | BLQ | BLQ | BLQ | BLQ | N/A | N/A |
| 3 SCD plasma | 44.56 | 2 | 50.13 | 5 | 5.58 | 0.09 |
| 4 haemolysed | 119.62 | 0 | 139.63 | 2 | 20.01 | 0.32 |
| 5 haemolysed | 27.59 | 0 | 33.06 | 2 | 5.47 | 0.09 |
| 6 haemolysed | 15.04 | 1 | 17.99 | 3 | 2.95 | 0.05 |
| 7 normal serum | 35.72 | 4 | 40.13 | 3 | 4.41 | 0.07 |
| 8 normal serum | 65.81 | 3 | 71.83 | 9 | 6.01 | 0.10 |
| 9 normal serum | 68.56 | 1 | 74.63 | 3 | 6.08 | 0.10 |

An increase above endogenous hemopexin:heme complex concentration (A) could be detected in all individuals when spiked with hemopexin alone (B) (delta complex concentration was >0 ug/mL). While results demonstrated low endogenous concentrations of hemopexin:heme complex within the SOD serum and plasma individuals and the haemolysed individuals (with the exception of one donor), the addition of hemopexin did cause a slight positive delta complex concentration in all samples, leading to a positive accessible heme measurement.

### 12. Conclusions

The fit-for-purpose validation of the hemopexin:heme complex and hemopexin-accessible heme determination in human serum and plasma have indicated the assay is suitable for use as a quasi-quantitative for complex and relative trends for accessible heme in serum samples. The on plate range for quantification of hemopexin:heme complex has been defined as 50-1000 g/mL in 100% human serum (1-20 μg/mL on plate) with limit of detection down to 0.15 μg/mL (on-plate concentration, background subtracted) with a degree of uncertainty. The assay is selective for hemopexin complexed with heme, as opposed to free apo-hemopexin. The assay demonstrated acceptable inter-assay, intra-assay and inter-operator precision and accuracy. The determination of hemopexin-accessible heme follows the same results as the complex quantification aspect of the assay.

This ELISA-based assay is a fit-for-purpose assay to 1.) Quasi-quantitate hemopexin:heme complex and 2.) Determine the relative hemopexin accessible heme from hemopexin spiked serum samples. The assay may be run as a single component assay to measure hemopexin:heme complex concentration only or as a two part assay to measure hemopexin:heme complex concentration and hemopexin-accessible heme.

### 13. Definitions/Abbreviations

Conc Concentration
CV Co-efficient of variation
DF Dilution Factor
Hpx Hemopexin
Hpx:heme Hemopexin:heme complex
μg/mL Microgram per millilitre
OD Optical Density
SCD Sickle cell disease
UHPC ultra high positive control
HPC High positive control
MPC Medium positive control
LPC lower positive control
ULPC ultra low positive control

1.-17. (canceled)

18. A nucleic acid comprising a sequence encoding a signal sequence for secretion of a CD91 polypeptide linked to a fragment of CD91, wherein the fragment:
(i) comprises ligand binding domain III of CD91,
(ii) does not extend N-terminally into ligand binding domain II of CD91, and
(iii) does not extend C-terminally into ligand binding domain IV of CD91.

19. The nucleic acid of claim 18, wherein the encoded fragment consists of amino acids 1184-3292 of CD91, wherein the amino acid positions are relative to SEQ ID NO: 1.

20. The nucleic acid of claim 18, wherein the encoded fragment consists of amino acids 2481-2942 of CD91, wherein the amino acid positions are relative to SEQ ID NO: 1.

21. The nucleic acid of claim 18, wherein the encoded fragment consists of the amino acid sequence of SEQ ID NO: 2 or an amino acid sequence having at least 85% identity to SEQ ID NO: 2.

22. The nucleic acid of claim 18, wherein the encoded signal sequence consists of amino acids 1-19 of SEQ ID NO: 1.

23. The nucleic acid of claim 18, wherein the encoded signal sequence is linked to the encoded fragment by a linker comprising the amino acid sequence of SEQ ID NO: 5.

24. The nucleic acid of claim 18, wherein the encoded signal sequence is linked to the encoded fragment by:
(i) a linker and
(ii) a first tag for polypeptide purification.

25. A polypeptide encoded by the nucleic acid of claim 24.

26. The polypeptide of claim 25, wherein the fragment of CD91 consists of the amino acid sequence of SEQ ID NO: 2.

27. The polypeptide of claim 25, wherein the linker comprises the amino acid sequence of SEQ ID NO: 5 and/or the first tag for polypeptide purification comprises the amino acid sequence of SEQ ID NO: 6.

28. The polypeptide of claim 25, further comprising a second tag for polypeptide purification, wherein the second tag for polypeptide purification is C-terminal to the fragment of CD91.

29. The polypeptide of claim 28, wherein the second tag for polypeptide purification is a His-tag.

30. The polypeptide of claim 25, wherein the polypeptide comprises amino acids 20-503 of SEQ ID NO: 3.

31. The polypeptide of claim 25, wherein the polypeptide comprises the amino acid sequence of SEQ ID NO: 3.

32. An assay comprising a solid support comprising a coating reagent, wherein the coating reagent comprises the polypeptide of claim 25.

33. A method of separating a hemopexin:heme complex from a biological sample, comprising:
(i) contacting the polypeptide of claim 25 with the biological sample, wherein the hemopexin:heme complex binds to the polypeptide, and
(ii) separating the polypeptide bound to the hemopexin:heme complex from the biological sample.

34. The method of claim 33, wherein the method is performed in vitro.

35. The method of claim 33, wherein the method is performed ex vivo.

36. The method of claim 35, wherein the biological sample is a blood sample and the method comprises apheresis.

37. A method of blocking or decreasing CD91 receptor-mediated endocytosis in a subject, comprising administering to the subject an effective amount of the polypeptide of claim 25.


SEQUENCE LISTING (6 sequences)

SEQ ID NO: 1 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSQEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 2 - protein, 462 aa, Artificial Sequence
  CD91 polypeptide, ligand binding domain III
PCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSQEHKCNASSQFLCSSGRCVAEALLCNGQDDCGDSSDERGCHIN

SEQ ID NO: 3 - protein, 503 aa, Artificial Sequence
  CD91 polypetide
MLTPPLLLLLPLLSALVAAAIDAPDYKDDDDKKPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSQEHKCNASSQFLCSSGRCVAEALLCNGQDDCGDSSDERGCHINHHHHHHHH

SEQ ID NO: 4 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 5 - protein, 5 aa, Artificial Sequence
  Linker
AIDAP

SEQ ID NO: 6 - protein, 9 aa, Artificial Sequence
  Flag-tag
DYKDDDDKK